(19) United States
(12) Patent Application Publication
(10) Pub. No.: US 2025/0375166 A1
(43) Pub. Date: Dec. 11, 2025

(54) FILTERING A PHYSIOLOGICAL SIGNAL USING A PRESSURE SENSOR SIGNAL TO GENERATE A PHYSIOLOGICAL MEASUREMENT

(71) Applicant: Meta Platforms Technologies, LLC, Menlo Park, CA (US)

(72) Inventors: Peter Twiss, San Jose, CA (US); Darien Niamir, Redwood City, CA (US); Ravi Krishna Shaga, Fremont, CA (US)

(21) Appl. No.: 19/234,248

(22) Filed: Jun. 10, 2025

Related U.S. Application Data
(60) Provisional application No. 63/658,261, filed on Jun. 10, 2024.

(51) Int. Cl. A61B 5/00

(52) U.S. Cl. CPC A61B 5/725; A61B 5/681; A61B 5/6885; A61B 5/742

(57) ABSTRACT

A method of generating a motion-artifact compensated physiological signal is described. The method includes receiving a contact pressure signal from a pressure sensor coupled to a circuit board within a capsule of a wrist-wearable device donned by a user and receiving a physiological signal from a physiological sensor coupled to the circuit board. The method further includes in accordance with a determination, based on the contact pressure signal, that a first motion-artifact criteria is satisfied, determining, based on the contact pressure signal, first motion-artifact adjustments to the physiological signal, and generating, based on the first motion-artifact adjustments, a first motion-artifact compensated physiological signal. The method further includes determining a physiological measurement based on the first motion-artifact compensated physiological signal.

### RELATED APPLICATION

This application claims priority to U.S. Provisional Application Ser. No. 63/658,261, filed Jun. 10, 2024, entitled “Pressure Sensing For Physiological Measurements,” which is incorporated herein by reference.

### TECHNICAL FIELD

This relates generally to using a measurements from a pressure sensor to filter a physiological signal and generate a physiological measurement.

### BACKGROUND

Wrist-wearable devices, such as smart watches, fitness trackers, etc. are becoming increasingly common to track data associated with a user. Wrist-wearable devices may perform many functions, including performing physiological measurements, analyzing movement activities, analyzing sleep, etc. Such functions rely on sensors that are disposed in and/or on a wrist-wearable devices. It is important for physiological measurements and analysis to be accurate, for example, for user safety and health. However, with the increasing number of components and sensors disposed within wrist-wearable devices, signal interference is increasingly an issue. Accordingly, methods, systems, and media for providing accurate physiological measurements on wrist-wearable devices is desired.

As such, there is a need to address one or more of the above-identified challenges. A brief summary of solutions to the issues noted above are described below.

### SUMMARY

A method of generating an accurate physiological measurement on a wrist-wearable device by filtering a physiological signal using a contact pressure signal is disclosed. The contact pressure signal can be generated by a strain gauge which determines the displacement of the force exerted on the object the strain gauge is coupled to. This provides a more accurate measurement and filtering signal than other sensors within the capsule of a wrist-wearable device in specific circumstances because it measures a more realistic signal with respect to what changes are occurring with respect to the user's wrist as opposed to movement of the wrist-wearable device itself which is captured by an IMU coupled to the capsule of the wrist-wearable device.

In accordance with some embodiments, a non-transitory computer readable storage medium including executable instructions that, when executed by one or more processors, cause the one or more processors to perform or cause performance of one or more operations. The one or more operations include: (i) receiving a contact pressure signal from a pressure sensor coupled to a circuit board within a capsule of a wrist-wearable device donned by a user and (ii) receiving a physiological signal from a physiological sensor coupled to the circuit board. In accordance with a determination, based on the contact pressure signal, that first motion artifact criteria is satisfied, the one or more operations include: (i) determining, based on the contact pressure signal, first motion-artifact adjustments to the physiological signal, and generating, based on the first motion-artifact adjustments, a first motion-artifact compensated physiological signal. In accordance with a determination, based on the contact pressure signal, that second motion artifact criteria is satisfied, the one or more operations include: (i) determining, based on the contact pressure signal, second motion-artifact adjustments to the physiological signal, and (ii) generating, based on the first motion-artifact adjustments, a second motion-artifact compensated physiological signal. The one or more operations further include determining a physiological measurement based on the first motion-artifact compensated physiological signal or the second motion-artifact compensated physiological signal.

Instructions that cause performance of the methods and operations described herein can be stored on a non-transitory computer readable storage medium. The non-transitory computer-readable storage medium can be included on a single electronic device or spread across multiple electronic devices of a system (computing system). A non-exhaustive of list of electronic devices that can either alone or in combination (e.g., a system) perform the method and operations described herein include an extended-reality (XR) headset/glasses (e.g., a mixed-reality (MR) headset or a pair of augmented-reality (AR) glasses as two examples), a wrist-wearable device, an intermediary processing device, a smart textile-based garment, etc. For instance, the instructions can be stored on a pair of AR glasses or can be stored on a combination of a pair of AR glasses and an associated input device (e.g., a wrist-wearable device) such that instructions for causing detection of input operations can be performed at the input device and instructions for causing changes to a displayed user interface in response to those input operations can be performed at the pair of AR glasses. The devices and systems described herein can be configured to be used in conjunction with methods and operations for providing an XR experience. The methods and operations for providing an XR experience can be stored on a non-transitory computer-readable storage medium.

The devices and/or systems described herein can be configured to include instructions that cause the performance of methods and operations associated with the presentation and/or interaction with an extended-reality (XR) headset. These methods and operations can be stored on a non-transitory computer-readable storage medium of a device or a system. It is also noted that the devices and systems described herein can be part of a larger, overarching system that includes multiple devices. A non-exhaustive of list of electronic devices that can, either alone or in combination (e.g., a system), include instructions that cause the performance of methods and operations associated with the presentation and/or interaction with an XR experience include an extended-reality headset (e.g., a mixed-reality (MR) headset or a pair of augmented-reality (AR) glasses as two examples), a wrist-wearable device, an intermediary processing device, a smart textile-based garment, etc. For example, when an XR headset is described, it is understood that the XR headset can be in communication with one or more other devices (e.g., a wrist-wearable device, a server, intermediary processing device) which together can include instructions for performing methods and operations associated with the presentation and/or interaction with an extended-reality system (i.e., the XR headset would be part of a system that includes one or more additional devices). Multiple combinations with different related devices are envisioned, but not recited for brevity.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIGS. 1A-1B illustrates an example of a wrist-wearable device detecting one or more physiological measurements, in accordance with some embodiments.

FIGS. 2A-2B illustrates another example of a wrist-wearable device detecting one or more physiological measurements, in accordance with some embodiments.

FIGS. 3A-3C illustrate a cross-sectional side view and an exemplary top view of an example wrist-wearable device and capsule, in accordance with some embodiments.

FIG. 4 illustrates an exploded view of the capsule of the wrist-wearable device, in accordance with some embodiments.

FIG. 5 is an example system for utilizing contact pressure signals for filtering physiological signals to generate a physiological measurement, in accordance with some embodiments.

FIG. 6 illustrates example signals that may be used in conjunction with the system shown in FIG. 5, in accordance with some embodiments.

FIG. 7 illustrates an example scenario of determining band tightness, in some embodiments.

FIG. 8 shows an example method flow chart for generating a physiological measurement, in accordance with some embodiments.

FIGS. 9A, 9B, 9C-1, and 9C-2 illustrate example MR and AR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user's physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user's hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user's hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single-or double-finger tap on a table, on a user's hand or another finger, on the user's leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user's possession (e.g., smartphones, laptops, imaging devices, intermediary devices, and/or other devices described herein).

The input modalities as alluded to above can be varied and are dependent on a user's experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors (used interchangeably with neuromuscular-signal sensors); (iii) IMUs for detecting, for example, angular rate, force, magnetic field, and/or changes in acceleration; (iv) heart rate sensors for measuring a user's heart rate; (v) peripheral oxygen saturation (SpO₂) sensors for measuring blood oxygen saturation and/or other biometric data of a user; (vi) capacitive sensors for detecting changes in potential at a portion of a user's body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user's environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiography (ECG or EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

### Generating Physiological Measurements

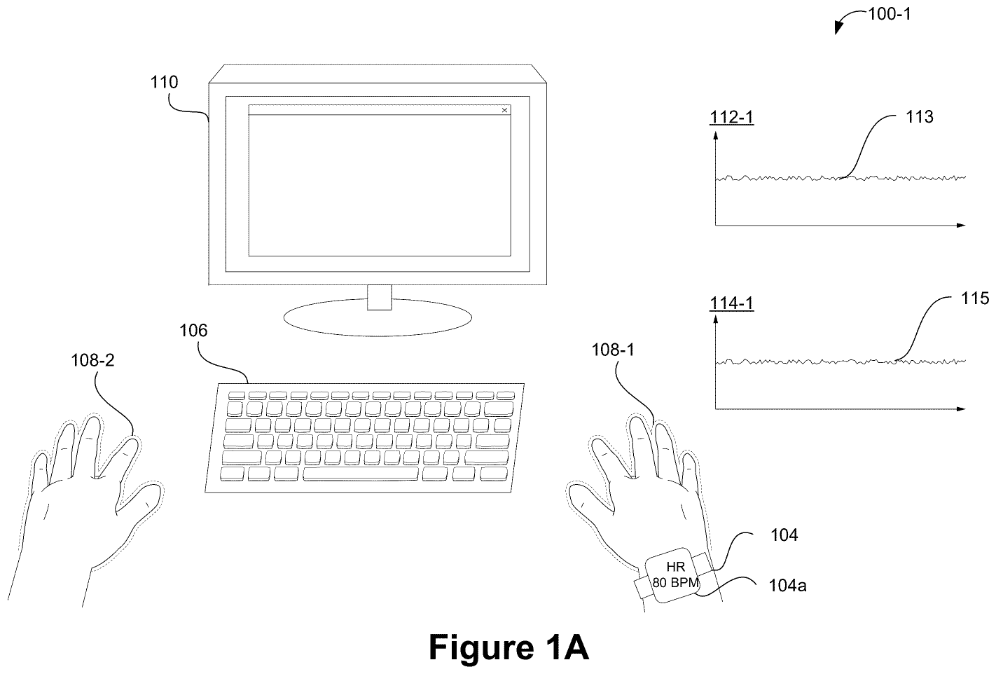

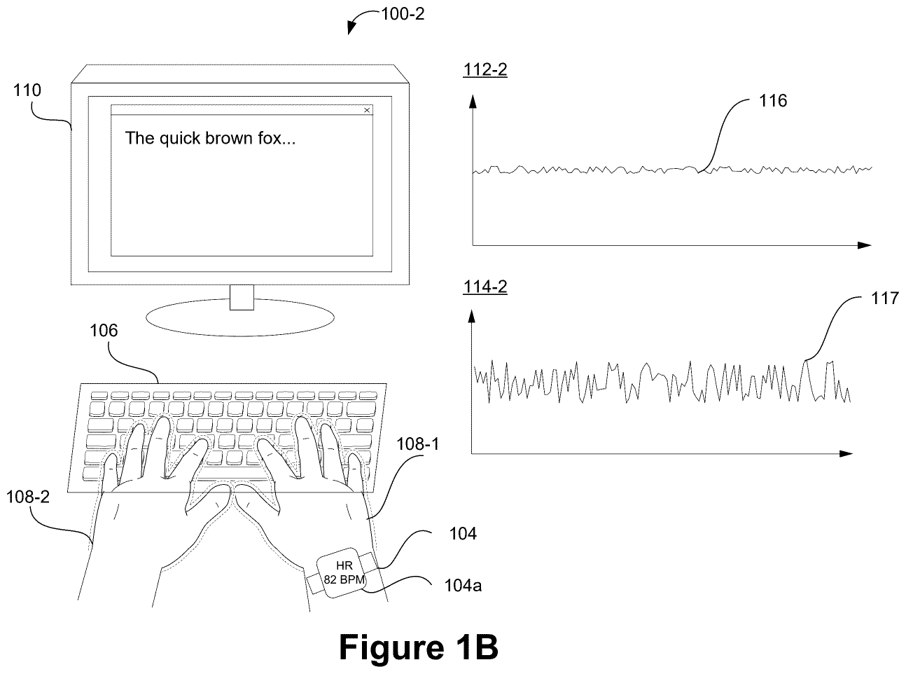

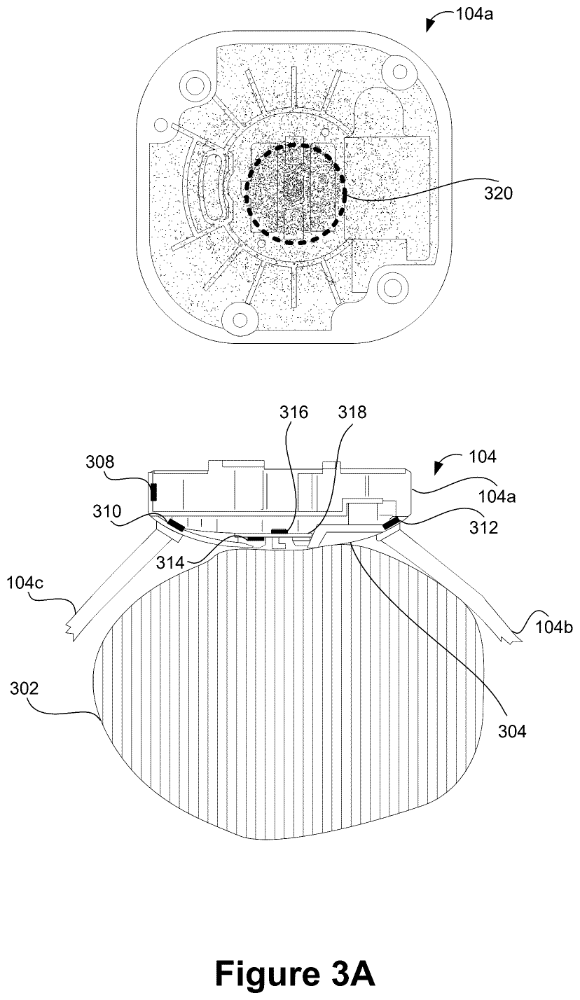

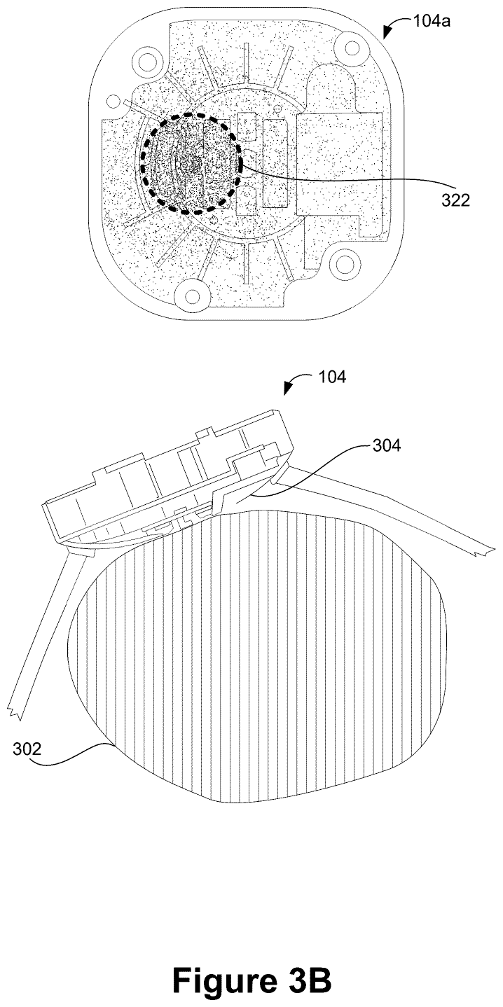

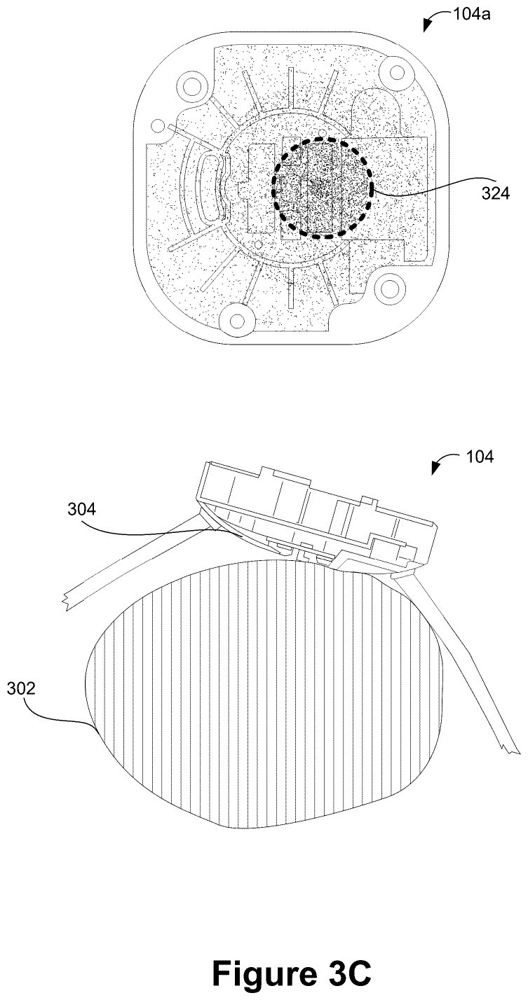

Wrist-wearable devices, such as smart watches and fitness trackers, include one or more sensors that are used to perform physiological measurements. Such physiological measurements may include photoplethysmography (PPG) based measurements (which may include heart rate, blood pressure, oxygen saturation, etc.), motion sensor based measurements (which may include respiration rate, etc.), electromyography (EMG) based measurements (which may include detection of muscle activity associated with the hand, wrist, arm, and/or fingers), body temperature measurements, or the like. The sensors may be disposed in and/or on the wrist-wearable device, for example, disposed in and/or proximate to a back cover (e.g., back cover 304; FIG. 3A) of the wrist-wearable device (e.g., wrist-wearable device 104; FIG. 1A) that is configured to be in contact with a wrist of the user, disposed in and/or proximate to a band of the wrist-wearable device that is configured to be in contact with the wrist of the wearer, or the like. For example, PPG-based measurements may be made using one or more light sources (e.g., light emitting diodes (LEDs)) and one or more light detectors that are positioned in and/or on a back cover of the wrist-wearable device such that light is emitted toward the wrist of the wearer and light reflected from tissue of the wearer is detected by the one or more light detectors. As another example, EMG-based measurements may be made using surface EMG electrodes disposed in and/or on the back cover of the wrist-wearable device, in and/or on the band of the wrist-wearable device, or the like.

Motion artifacts (e.g., signal noise/interference) may interfere with physiological measurements obtained using biosensors (which may include PPG sensors, EMG sensors, ultrasonic sensors, etc.). For example, the motion of a user moving their arm while exercising (e.g., running, performing aerobic activities, lifting weights, etc.) or while typing or performing other routine activities may interfere with heart rate calculations or other physiological measurements. Conventional techniques utilize an inertial measurement unit (IMU) disposed in the wearable device to measure, e.g., acceleration information. The IMU data may then be used to correct motion artifacts of biosensor signals. For example, IMU data may be used to correct motion artifacts in PPG signals. However, conventional techniques which utilize IMU data may be inaccurate, particularly for certain types of user movements. For example, the accelerometer may not accurately detect user motion during certain types of exercise (e.g., high intensity interval training), while typing, etc., and accordingly, the motion artifacts may not sufficiently correct the biosensor signals. This leads to inaccuracies in the physiological measurements, because the biosensor signal itself has motion artifacts.

Disclosed herein are techniques for using contact pressure information that indicates a pressure between a back cover of a wrist-wearable device and the wrist of the wearer to correct motion artifacts. Because the contact pressure corresponds to the tightness of the band (e.g., the contact pressure will increase as band tightness increases), contact pressure is sometimes referred to hercin as a “band tightness indicator,” or “BTI.” The contact pressure signal may indicate user motion, e.g., as the pressure of the user's wrist changes with activity against the back capsule of the wrist-wearable device. This contact pressure signal may be more accurate for motion artifacts for certain activities than the IMU data. For example, the contact pressure signal may vary substantially as the user types, e.g., due to the wrist extending and flexing and/or due to finger movements, which may be accurately captured in contact pressure signals but not reflected in IMU data. For such activities, the contact pressure signal may more accurately correct biosensor signals than IMU data.

The techniques described herein use pressure sensors disposed in and/or on the wrist-wearable device to determine a contact pressure between a back cover of the wrist-wearable device to the wrist of the wearer. The pressure sensors may be strain gauge type sensors that detect deflection or bending of a surface the strain gauge type sensor is affixed to. The deflection/bending is detected based on the contact pressure and/or compression force type sensors that detect a compression force between two surfaces the compression force type sensor is affixed to. Types and locations of pressure sensors are described in more detail in connection with FIGS. 1A-4.

It should be noted that the term “contact pressure” as used herein generally refers to a measure of force of a portion of a device surface (e.g., a back cover of a wrist-wearable device) on an area of body surface (e.g., a wrist of a wearer of the wrist-wearable device). As used herein, a “pressure sensor” may include a pressure sensor which measures force per unit area (e.g., pounds per square inch, or the like), or a force sensor which measures a force. In instances in which a force is measured, a measure of contact pressure may be determined based on the measured force, for example, by dividing the measured force by a known surface area (e.g., an area of the back cover of the wrist-wearable device, or the like).

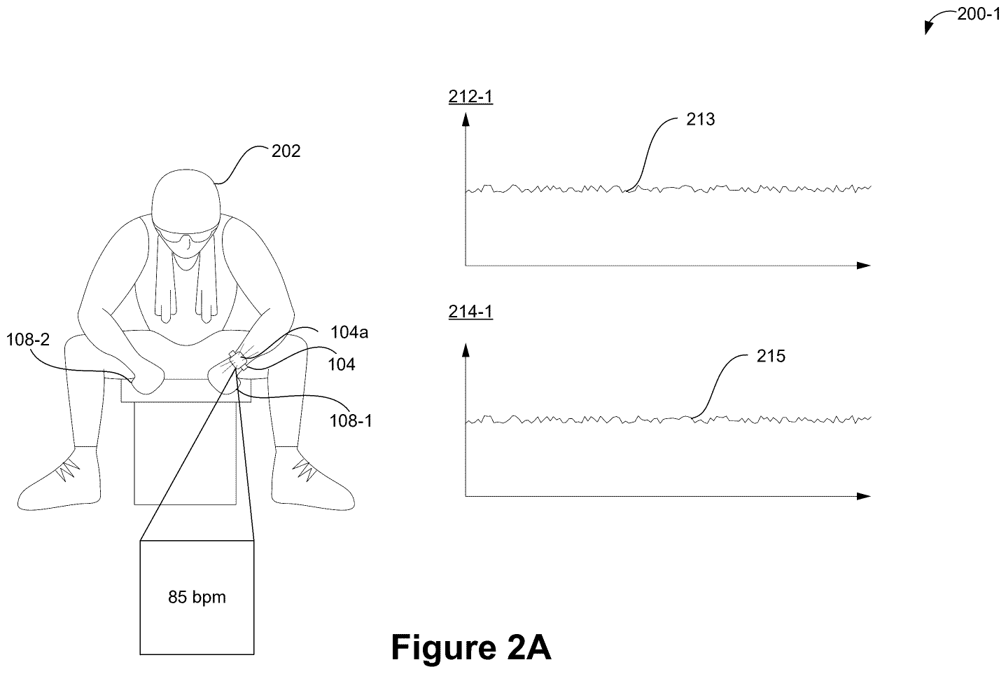

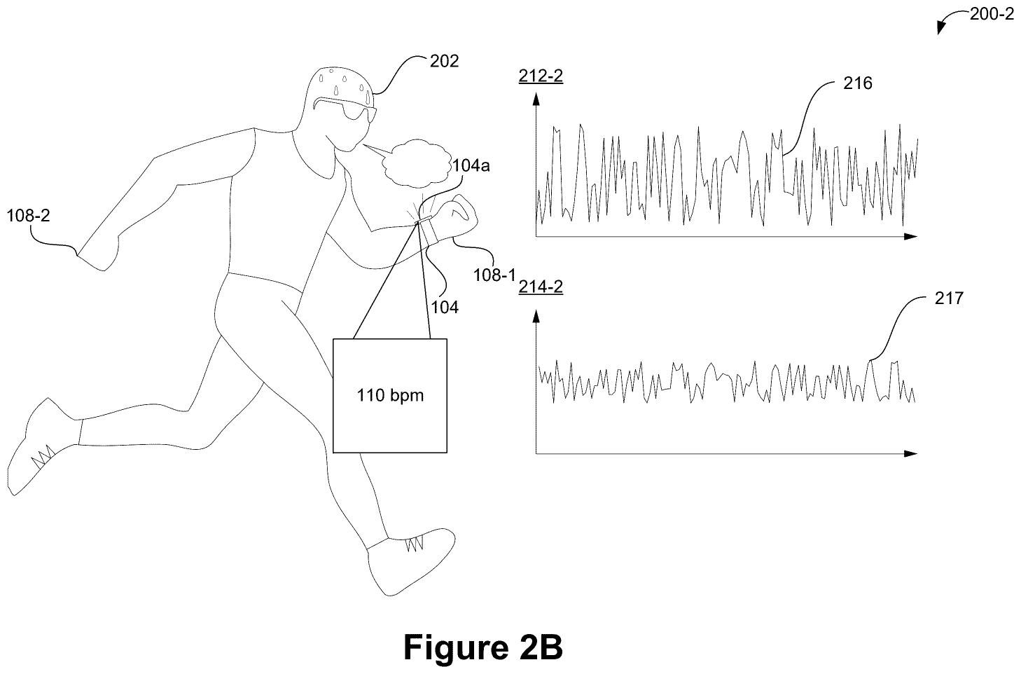

FIGS. 1A-1B illustrates an example of a wrist-wearable device detecting one or more physiological measurements, in accordance with some embodiments. FIG. 1A illustrates a wrist-wearable device 104 worn by a user detecting one or more physiological measurements at a first point in time. The scene 100-1 illustrates the user wearing the wrist-wearable device 104 on the user's right hand 108-1 prior to typing on a keyboard 106 with the user's hands 108 (e.g., the user's right hand 108-1 and the user's left hand 108-2). The wrist-wearable device 104 can be worn on either wrist of the user; FIG. 1A-2B illustrates the user wearing it on the wrist of their right hand 108-1 for example purposes. The monitor 110 is configured to illustrate a message that is typed by the user. In some embodiments, the physiological measurements are displayed on the capsule 104a of the wrist-wearable device 104. FIG. 1A further illustrates an example resting heart rate of the user (e.g., 80 beats per minute (bpm)) displayed while the user is at rest prior to typing.

Graph 112-1 illustrates the IMU signal 113, captured by an IMU sensor of the wrist-wearable device 104, at a first point in time while the wrist-wearable device 104 is measuring the one or more physiological signals. At the first point in time the user is at rest prior to typing and the IMU signal 113 is substantially steady and includes relatively low noise.

Graph 114-1 illustrates the contact pressure signal 115, captured by a pressure sensor of the wrist-wearable device 104, at a first point in time while the user is at rest prior to typing. The contact pressure signal 115, like the IMU signal 113, is substantially steady at the first point in time as the user is at rest. For example, because movement of capsule 104a, as detected by an IMU, does not substantially change and contact pressure, as detected by the pressure, exerted between the user's wrist and the wrist-wearable device 104 does not substantially change, both the IMU signal 113 and the contact pressure signal 115 are substantially steady.

The IMU signal 113 and the contact pressure signal 115 can be used to indicate that the user is not substantially moving their hand and/or wrist because there is not a high level of activity in the IMU data or a substantial change in the contact pressure data.

FIG. 1B illustrates the wrist-wearable device 104 detecting one or more physiological measurements at a second point in time. The scene 100-2 includes the user typing on the keyboard 106 and the monitor 110 displaying the message typed by the user (e.g., the quick brown fox . . . ). While the user is moving their right hand 108-1 to type, the capsule 104a of the wrist-wearable device may not move substantially whereas a contact pressure exhibited between the user's wrist and the backplate of the wrist-wearable device 104 (e.g., back cover 304 of the wrist-wearable device 104; FIG. 3A) may change substantially. Because the capsule 104a of the wrist-wearable device may not move substantially while the user is typing, the IMU of the wrist-wearable device 104 may not detect the user's movements which can introduce motion artifacts into the physiological measurements.

Graph 112-2 illustrates the IMU signal 116 at a second point in time while the user is actively typing. Although the user is typing, the IMU signal 116 has not substantially changed from the IMU signal 113 at a first point in time. In some embodiments, the IMU is unable to detect certain movements with the same level of precision as the contact pressure signal during certain activities such as typing as illustrated in FIG. 1A and FIG. 1B. Using only the IMU signal 116 to filter the physiological signal before generating the physiological measurements can result in inaccurate physiological measurements as the IMU is unable to detect the motion artifacts in all user movements or activity (e.g. typing).

Graph 114-2 illustrates the contact pressure signal 117 at a second point in time. The contact pressure signal 117 illustrates the motion artifacts representative of the user's movements in their wrist (e.g., the user's wrist flexing while typing) while typing. The contact pressure signal 117 can be used to filter the motion artifacts out of the physiological signals prior to generating the physiological measurement, which results in accurate physiological measurements. The physiological measurement can be displayed on the capsule 104a of the wrist-wearable device 104 (e.g., the heart rate (HR). For example, if only the IMU signal 116 was used to the filter the physiological measurement, the slight increase in the physiological measurement (e.g., the heart rate increasing from 80 bpm in FIG. 1A to 82 bpm in FIG. 1B) possibly would not have been detected.

FIGS. 2A-2B illustrates another example of a wrist-wearable device detecting one or more physiological measurements, in accordance with some embodiments. FIGS. 2A-2B further illustrate a scenario where the user 202 is engaged in a high intensity activity (e.g., running). While the user in engaged in a high intensity activity, an IMU of the wrist-wearable device detects a substantial amount of movement that that may not be filtered out of the physiological signal. Movement that is not filtered out of the physiological signal may incorrectly alter the physiological measurement if the (fully or partially) unfiltered physiological signal is used during the physiological measurement determination.

FIG. 2A further illustrates a scene 200-1 including the user 202, at a first point in time, sitting before starting their run. In some embodiments, the user 202 is wearing a wrist-wearable device 104 on their right hand 108-1. In some embodiments, the physiological measurement (e.g., the user's resting heart rate at 85 bpm) is displayed on the capsule 104a of the wrist-wearable device 104.

Graph 212-1 illustrates the IMU signal 213, as measured by an IMU sensor of the wrist-wearable device 104, at a first point in time while the wrist-wearable device 104 is measuring the one or more physiological signals. During the first point in time the user 202 is at rest prior to running and the IMU signal 213 is steady and includes relatively low noise.

Graph 214-1 illustrates the contact pressure signal 215, as measured by a pressure sensor of the wrist-wearable device 104, at a first point in time while the user 202 is at rest prior to running. The contact pressure signal 215, like the IMU signal 213, is substantially steady at the first point in time. The wrist-wearable device 104 can use contact pressure signal 215 and the IMU signal 213 to determine that the user 202 is not moving their hand and/or wrist substantially as the IMU signal 213 does not show a high level of activity and/or there is no substantial change in the contact pressure signal 215.

FIG. 2B illustrates a scene 200-2 including the user 202 running at a second point in time. As the user 202 is running, their arms and hands 108 are moving swiftly. Thus, the IMU within the capsule 104a of the wrist-wearable device 104 is moving and generating a large and noisy signal proportional to the user's 202 movements. In contrast, the contact pressure between the user's wrist and the wrist-wearable device 104 is changing a smaller amount, but still changing slightly.

Graph 212-2 illustrates the IMU signal 216, captured by an IMU sensor of the wrist-wearable device 104, at a second point in time while the wrist-wearable device 104 is measuring the one or more physiological signals. During the second point in time the user 202 is running and the IMU signal 216 is large (e.g., has a large amplitude) and noisy. The IMU signal 216 is proportional to the significant movement engaged in by the user's right hand 108-1.

Graph 214-2 illustrates the contact pressure signal 217, captured by a pressure sensor of the wrist-wearable device 104, at a second point in time while the user 202 is running. The contact pressure signal 217 has increased in amplitude compared to the contact pressure signal 215 at the first point in time, however, the contact pressure signal 217 has not increased in amplitude and/or frequency as much as the IMU signal 216. Furthermore, the delta in the change between the contact pressure signal 217 and the IMU signal 216 indicates the user's right hand 108-1 is moving substantially but the user's wrist is not flexing substantially. From the first point in time to the second point in time, the IMU signal 216 shows a greater change because the user's hand 108-1 is moving substantially. However, the contact pressure signal 217 shows a smaller change compared to the first point in time because the user 202 is not performing movements that alter the contact pressure between the user's wrist and the capsule 104a substantially. The IMU signal 216 is less reliable as a filtering signal for the final physiological measurement because the signal is too large and not as accurate when compared to the motion artifacts affecting the physiological signal detected by the wrist-wearable device 104. Thus, the contact pressure signal 217 is more accurate when used for filtering the physiological signal to determine the final physiological measurement.

FIGS. 3A-3C illustrate a cross-sectional side view and an exemplary top view of an example wrist-wearable device and capsule, in accordance with some embodiments. As illustrated in FIG. 3A, a back cover 304 (sometimes referred to herein as a “bottom portion,” “a bottom cover portion,” or a “back cover portion”) rests on a body portion (e.g., the user's wrist 302) of a body of a wearer (e.g., a wrist surface, an arm surface, or the like). Wrist-wearable device 104 includes two band portions 104c and 104d, each of which are coupled to an end of a capsule 104a (e.g., via clips, hinges, an adhesive, or the like).

A top portion of capsule 104a may include a display screen, and a back cover 304 rests on body portion (e.g., the user's wrist 302). One or more pressure sensors (e.g., a strain gauge, MEMs based pressure sensor, etc.) may be affixed to and/or embedded within capsule 104a. Example locations of pressure sensors are depicted in FIG. 3A. For example, a pressure sensor 308 is positioned at a side of capsule 104a. As another example, pressure sensors 310 and 312 are positioned along a bottom portion of capsule 104a, each of pressure sensors 310 and 312 being proximate to an end of capsule 104a at which band portion 104b or band portion 104c is coupled. In some embodiments, pressure sensor 314 is positioned along a bottom portion of capsule 104a proximate to back cover 304. As still another example, pressure sensor 316 is positioned on a chip or printed circuit board (PCB) 318 disposed within capsule 104a. In some implementations, PCB 318 may include one or more sensors suitable for collecting data for performing physiological measurements, such as one or more light-emitting diodes (LEDs), one or more light detectors, one or more accelerometers, one or more gyroscopes, or the like. In some implementations, light from light emitters may shine through back cover 304 toward body portion (e.g., the user's wrist 302), and light reflected from body portion (e.g., the user's wrist 302) or a region of the body proximate to body portion (e.g., the user's wrist 302) may be transmitted through back cover 304 and captured by one or more light detectors within capsule 104a. It should be noted that although five pressure sensors are depicted in FIG. 3A, this is merely exemplary, and, in some implementations, a wrist-wearable device may include any suitable number of pressure sensors (e.g., one, two, three, four, six, ten, or the like).

In some embodiments, the pressure sensors are strain gauge sensors. In some embodiments, a pressure sensor 314 is affixed via an adhesive layer to the back cover 304 of the wrist-wearable device. For example, pressure sensor 314 may be affixed to a first side of the back cover 304 (e.g., in an interior portion of a capsule 104a of the wrist-wearable device 104), and a second side (e.g., opposing) back cover 304 may be configured to be in contact with a body portion of the user (e.g., a wrist of the user). In some embodiments, a pressure sensor may be affixed to a surface of which no side is configured to be in contact with the wearer. For example, in some embodiments, pressure sensor 308 may be affixed to a side portion of a capsule 104a.

In some implementations, a pressure sensor may be a compression force sensor. A compression force sensor may be positioned between two surfaces and may be configured to detect a compression force between the two surfaces. For example, a compression force sensor may be positioned between a back cover 304 of a wrist-wearable device 104 and another surface of the wrist-wearable device 104 (e.g., a PCB surface within an interior of a capsule 104a of the wrist-wearable device 104) such that the signal produced by the compression force sensor is directly proportional to a force of the back cover 304 of the wrist-wearable device 104 on a body portion of the user (e.g., the user's wrist 302).

In some embodiments, pressure sensor 316 includes a compression force sensor in accordance with some embodiments. Pressure sensor 316 may be affixed to a rubber element, which may in turn be affixed via adhesive the back cover 304. The adhesive be on a first side of back cover 304 (e.g., in an interior portion of a capsule 104a of the wrist-wearable device 104), and a second (e.g., opposing) side of the back cover 304 may be configured to be in contact with a body portion of the wearer (e.g., the user's wrist 302). In some embodiments, the rubber element may serve to thermally and/or mechanically isolate pressure sensor 316 such that pressure sensor 316 is not affected by thermal variations on the portion of back cover 304 that is physically in contact with the skin of the user. An opposing side of pressure sensor 316 can be affixed to an opposing surface. The opposing surface may be positioned within an interior of a capsule 104a of the wrist-wearable device 104. For example, the opposing surface may be a surface of a PCB 318 within the capsule 104a.

As described above, multiple (e.g., two, three, four, five, six, etc.) pressure sensors may be disposed proximate to a back cover of a wrist-wearable device 104 such that the pressure sensors (e.g., pressure sensors 308-316) are configured to detect variations in pressure across an X-Y plane corresponding to the back cover of the wrist-wearable device. The variations in pressure across the X-Y plane may be used to detect a tilt of a capsule of the wrist-wearable device relative to a body (e.g., a wrist surface) of the user. For example, the variations in pressure across the X-Y plane may detect that the capsule is tilted to one side (e.g., due to the wrist-wearable device being too big or too small for the wearer). In some implementations, variations in pressure across the X-Y plane may be used to detect where on a top surface (e.g., a display) of the capsule the wearer is pressing or moving a finger. In some embodiments, such pressure variations may be used as user input, for example, on a user interface (e.g., to scroll in a user interface presented on the display, to adjust a volume of audio content being presented by a user device paired to the wrist-wearable device, or the like).

For example, the capsule 104a is illustrated in FIG. 3A including a heat map including a plurality of dots indicating the center of the contact pressure based on measurements from multiple pressure sensors. This further illustrates the pressure sensor measuring strain transfer from the back cover 304 to the sensor board. FIG. 3A further illustrates the wrist-wearable device 104 placed on the user's wrist 302 in such a manner that the capsule 104a is aligned with the top of the user's wrist 302 such that the contact pressure exerted is centered in the middle of the capsule 104a as illustrated with location 320.

FIG. 3B illustrates the wrist-wearable device 104 offset on the user's wrist 302 such that the contact pressure between the user's wrist 302 and the back cover 304 is altered and centered at a different portion of the capsule 104a. For example, the capsule 104a illustrated in FIG. 3B shows that based on the measurements generated by the multiple pressure sensors the center of the contact pressure is located toward the edge of the capsule at location 322.

FIG. 3C illustrates the wrist-wearable device 104 offset on the user's wrist 302 such that the contact pressure between the user's wrist 302 and the back cover 304 is altered and centered at a different portion of the capsule 104a. For example, the capsule 104a illustrated in FIG. 3B shows that based on the measurements generated by the multiple pressure sensors the center of the contact pressure is located toward another edge of the capsule at location 324.

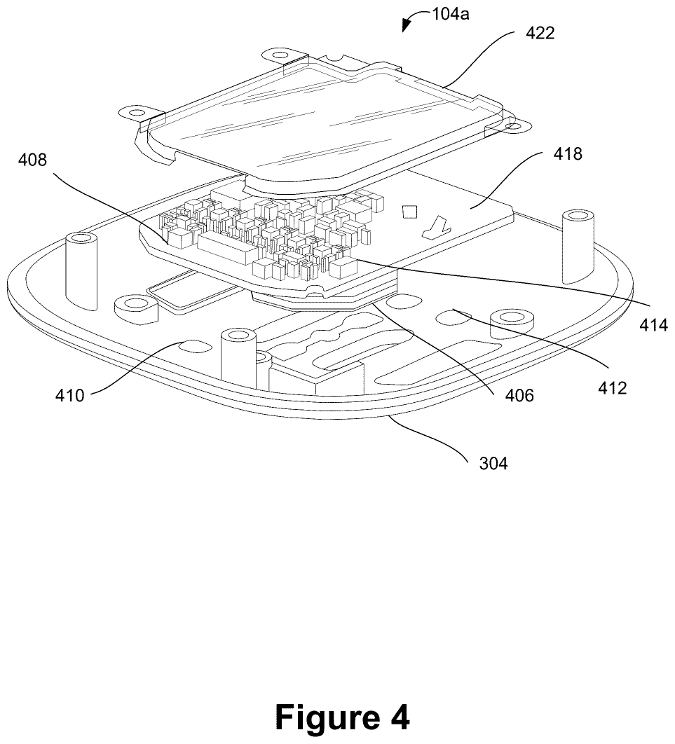

FIG. 4 illustrates an exploded view of the capsule of the wrist-wearable device, in accordance with some embodiments. A cover 422 configured to protect printed circuit board (PCB) 418, optical module 406, and the back cover 304 are illustrated in the exploded view. Furthermore, multiple pressure sensors are illustrated. As discussed in FIG. 3A, the wrist-wearable device includes one or more pressures sensors such as pressure sensor 408 coupled to the PCB 418 (e.g., the sensor board). In some embodiments, the PCB 418 is one of a plurality of PCBs or circuit boards of the wrist-wearable device 104. FIG. 3A further illustrated multiple pressure sensors disposed proximate to the back cover. In the example shown in FIG. 4, exemplary pressure sensors 408 and 414 are each affixed to a printed circuit board (PCB), each of which is disposed in a different location proximate to the back cover and exemplary pressure sensors 410 and 412, and are disposed on a common PCB which is in turn disposed proximate to the back cover. In some implementations, the PCB 418 may additionally include other sensors and/or one or more processors which may be used for performing physiological measurements, and/or for any other suitable purposes.

As discussed briefly above, a pressure sensor may be a strain gauge type sensor. A strain gauge type sensor may detect bending due to pressure of a back cover 304 of the wrist-wearable device 104 on a body portion of the wearer. For example, a strain gauge sensor positioned proximate to the back cover may detect a bending or a deflection of the back cover of the wrist-wearable device. As another example, a strain gauge sensor positioned proximate to a side of a capsule of the wrist-wearable device may detect a bending or deflection of the side of the capsule, due to, e.g., the band of the wrist-wearable device pulling on the side of the capsule. In some embodiments, a strain gauge type sensor may be affixed to a surface of which the strain gauge type sensor detects deflection or bending.

Wrist-wearable devices, such as fitness trackers or smart watches, frequently measure physiological characteristics of a wearer. These physiological characteristics may include heart rate, oxygen saturation, blood pressure, or the like. These physiological characteristics may be determined using measurements from one or more sensors on-board the wrist-wearable device, such as one or more light emitters, one or more light detectors, one or more accelerometers, one or more gyroscopes, etc. By way of example, PPG is an example of a technique that may be used to determine heart rate, oxygen saturation, blood pressure, etc. In PPG, light is emitted toward skin of the wearer, and the light is then reflected from the skin and/or from various internal body regions (e.g., blood vessels, blood cells, bone, etc.). The reflected light is then captured by one or more light detectors of the wrist-wearable device, and characteristics of the reflected light, such as changes in absorption over different wavelengths of light, may be used to determine heart rate, oxygen saturation, blood pressure, etc.

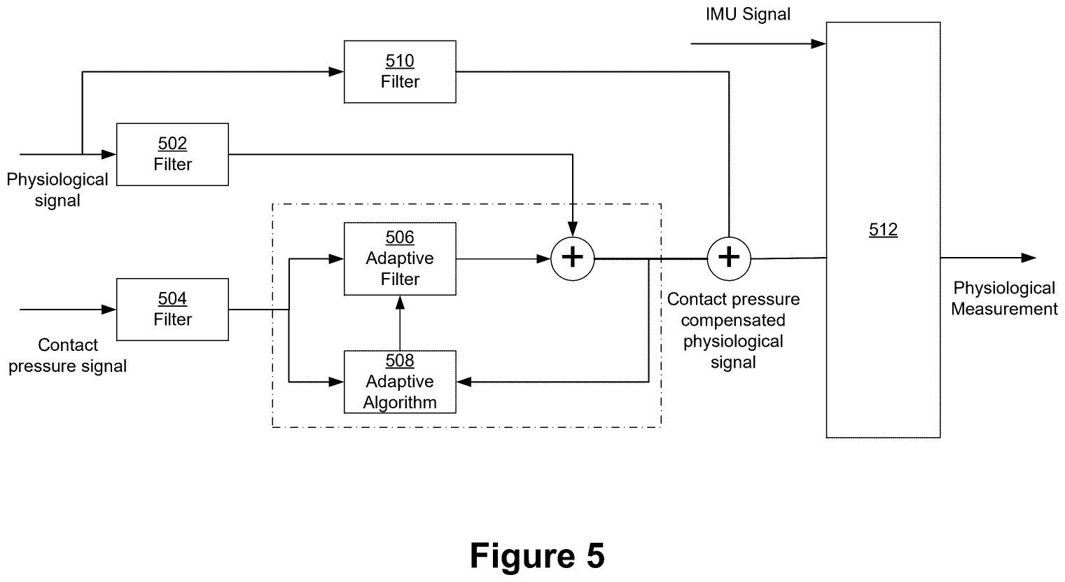

FIG. 5 is an example system for utilizing contact pressure signals for filtering physiological signals to generate a physiological measurement, in accordance with some embodiments. As illustrated, contact pressure signals, referred to in FIGS. 1A-2B as “BTI signals,” are obtained, e.g., from one or more pressure sensors disposed in or on the wearable device. Concurrently, PPG signals (e.g., physiological signals) may be obtained, e.g., using one or more LEDs and/or detectors disposed in or on the wearable device. In some embodiments, the physiological signal is processed using a filter 502 and the contact pressure signal is filtered using filter 504. In some embodiments, filter 502 and 504 are band pass filters.

Following band-pass filtration at filter 504, the contact pressure signals may be used to determine weights for an adaptive filter 506. The adaptive filter 506 is in turn used to filter the physiological signal. In other words, the contact pressure signal is used to determine how the physiological signals are filtered. For example, increased amplitude of the contact pressure signals may cause more aggressive filtration of the physiological signals. The weights of the adaptive filter 506 may be dynamically updated over time, reflecting changes in the contact pressure signals as the user moves. For example, as illustrated in FIG. 2B, as the user 202 runs the contact pressure signals are continually collected and update the adaptive filter 506 to provide an accurate weighting to the adaptive filter 506 in real time. In some embodiments, the contact pressure signal includes one or more motion artifacts which are used to adjust the physiological signal to provide a cleaner signal for processing.

The adaptive algorithm 508 is part of the adaptive filter 506 and includes filter architectures to cancel noise. The adaptive filter 506 and the adaptive algorithm 508 continuously receive the filtered contact pressure signals and the filtered physiological signals and adjusts what the adaptive filter filters out of the signal. For example, a PPG signal (e.g., an example physiological signal) is corrupted by motion, cardiac noise, etc. The contact pressure signal represents the noise signature. The adaptive algorithm 508 and adaptive filter 506 receive the filtered physiological and contact pressure signals such that the noise represented by the contact pressure signal is removed from the physiological signal to generate a clean physiological signal.

As indicated in FIG. 5, The physiological signal is filtered using filter 510 which includes a low pass filter and with the output of the adaptive algorithm 508 generates the filtered PPG signal (referred to in FIG. 5 as “contact pressure compensated physiological signal” may be provided to an algorithm 512 configured to generate a physiological measurement using the filtered physiological signal. For example, the physiological measurement may be a heart rate of the user. Other examples include an oxygen saturation, a blood pressure, etc. In some embodiments, the algorithm 512 may additionally take as input IMU signals. In other words, the algorithm 512 may separately consider IMU data, e.g., for further motion artifact suppression, although this is optional.

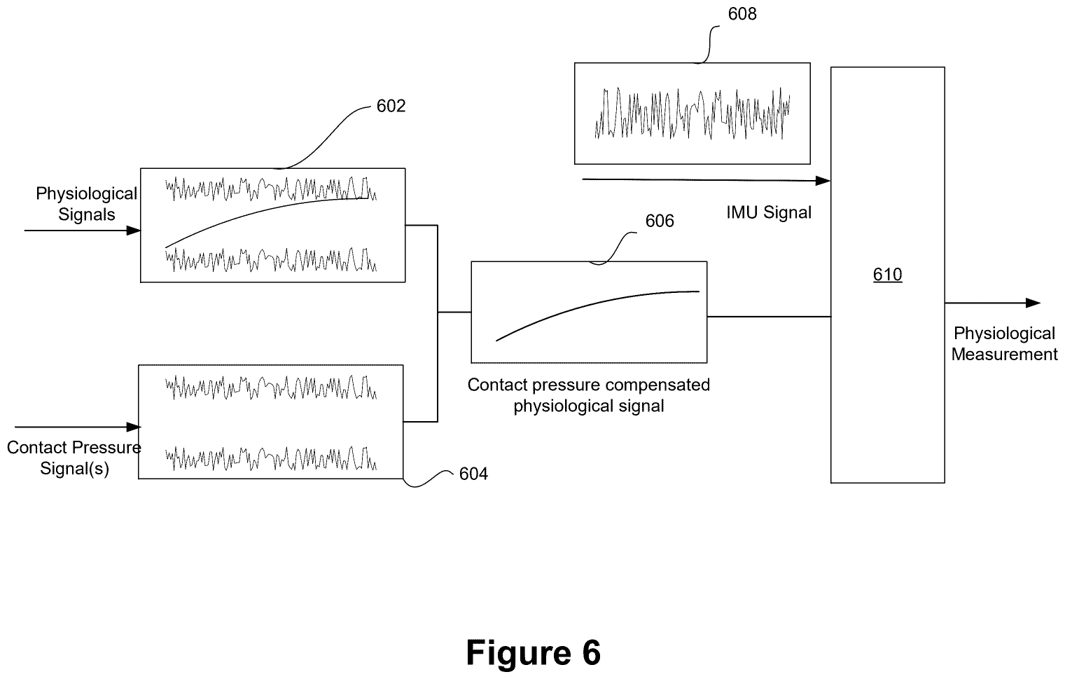

FIG. 6 illustrates example signals that may be used in conjunction with the system shown in FIG. 5, in accordance with some embodiments. As illustrated, physiological signals 602 and contact pressure signals 604 may be used to generate a motion artifact corrected physiological signal 606, which is provided to algorithm 610. Algorithm 610 also takes, as input, IMU signals 608, which may be used for further motion artifact compensation.

Although FIGS. 5 and 6 illustrate use of contact pressure signals 604 to correct motion artifacts in physiological signals, the contact pressure signals 604 may be used to correct motion artifact in any type of biosensor signal. Other examples include EMG signals, IPG signals, ultrasound signals, etc. Additionally, although FIG. 5 illustrates use of contact pressure signals to adaptively filter a biosensor signal, in some embodiments, adaptive filter weights may be determined based on a combination of contact pressure signals and IMU data. In some embodiments, the signal inputs used to determine adaptive filter weights may be selected based on context. For example, in instances in which a user provides input initiating a particular type of activity (e.g., a particular type of exercise), the signal inputs may be selected based on the user input. As a more particular example, responsive to a user indicating they are beginning a high intensity interval training type exercise, motion artifacts may be corrected using contact pressure signals (with or without consideration of IMU data), whereas for other types of activity, contact pressure signal may not be considered.

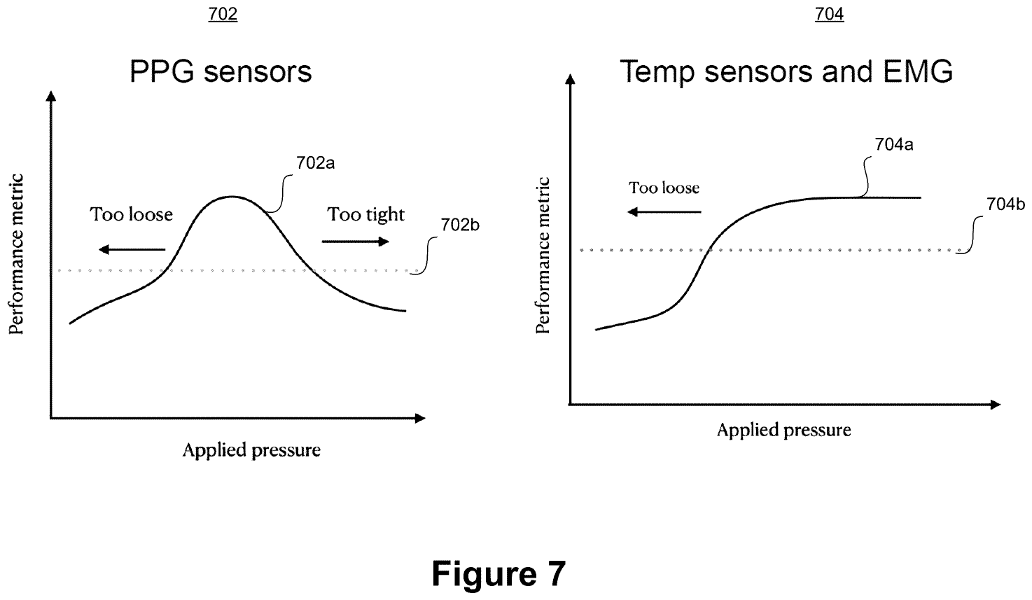

FIG. 7 illustrates an example scenario of determining band tightness, in some embodiments. In some embodiments, the contact pressure signal(s) may be used to determine a quality of the contact of the wrist-wearable device 104 to the skin of the user. The performance of all health sensors (e.g., PPG, skin temperature sensors, EMG, etc.) on wrist-wearables are sensitive to band fit and quality of contact to the back cover to the skin. Determining the quality of contact of the wrist-wearable device 104 and indicating to the user if the contact is not optimized for performance will help the user receive more accurate data. For example, as shown in FIG. 7, graph 702 illustrates a waveform 702a that compares the applied pressure on the x-axis to the performance metric on the y-axis for PPG sensors. For example, as illustrated in graph 702, when the applied pressure (e.g., contact pressure) is too low and the performance metric is below a threshold amount (e.g., threshold 702b), the band of the wrist-wearable device is too loose. In some embodiments, the system notifies the user via a message on the display of the wrist-wearable device or in another method to indicate to the user that they need to tighten the band for the best results. As illustrated in graph 702, when the applied pressure is high and the performance metric is below the threshold amount, the band of the wrist-wearable device is too tight. FIG. 7 also illustrates graph 704 which illustrates a waveform 704a that compares applied pressure to performance metrics for temperature sensors and EMG sensors. For example, as illustrated in graph 704, when the applied pressure is too low, the performance metric is below a threshold amount 704b, the band of the wrist-wearable device is too low.

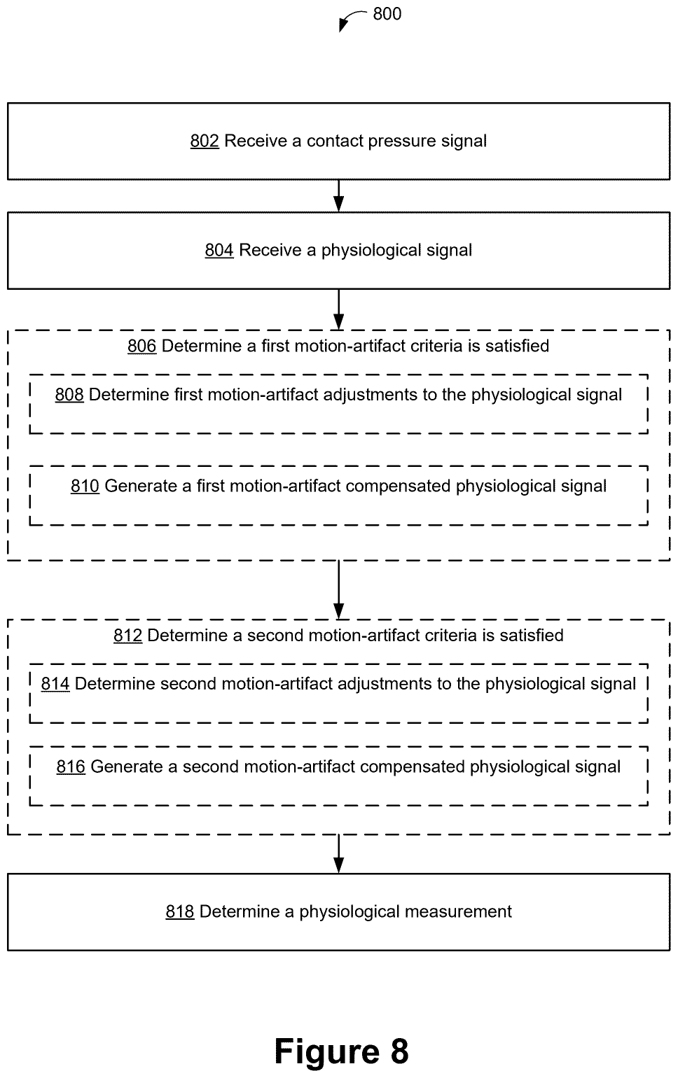

FIG. 8 illustrates a flow diagram of a method of generating a physiological measurement, in accordance with some embodiments. Operations (e.g., steps) of the method 800 can be performed by one or more processors (e.g., central processing unit and/or MCU) of a system including at least a wrist-wearable device (e.g., wrist-wearable device 104; FIG. 1A). At least some of the operations shown in FIG. 8 correspond to instructions stored in a computer memory or computer-readable storage medium (e.g., storage, RAM, and/or memory) of a wrist-wearable device (e.g., wrist-wearable device 104; FIG. 1A). Operations of the method 800 can be performed by a single device alone or in conjunction with one or more processors and/or hardware components of another communicatively coupled device (e.g., wrist-wearable device 104; FIG. 1A) and/or instructions stored in memory or computer-readable medium of the other device communicatively coupled to the system. In some embodiments, the various operations of the methods described herein are interchangeable and/or optional, and respective operations of the methods are performed by any of the aforementioned devices, systems, or combination of devices and/or systems. For convenience, the method operations will be described below as being performed by particular component or device, but should not be construed as limiting the performance of the operation to the particular device in all embodiments.

A method of generating an accurate physiological measurement on a wrist-wearable device by filtering a physiological signal using a contact pressure signal is disclosed. (A1) FIG. 8 shows a flow chart of a method 800 of generating a physiological measurement, in accordance with some embodiments. The method 800 occurs at a wrist-wearable device (e.g., wrist-wearable device 104; FIG. 1A) with one or more of a capsule (e.g., capsule 104a; FIG. 1A), pressure sensors (e.g., pressure sensors 308-314; FIG. 3A), a display, etc. In some embodiments, the method 800 includes, receiving (802) a contact pressure signal (e.g., contact pressure signals 115, 117, 215, 217; FIGS. 1A-2B and FIGS. 5-6). In some embodiments, the contact pressure signal is generated via a pressure sensor (e.g., pressure sensors 308-314; FIG. 3A) coupled to the circuit board (e.g., PCB 318; FIG. 3A) within a capsule (e.g., capsule 104a; FIG. 1A) of a wrist-wearable device donned by a user. As discussed in FIGS. 1A-6, the pressure sensors can include strain gauge sensors coupled to the PCB 318 and/or the (e.g., back cover 304; FIG. 3A) of the wrist-wearable device.

In some embodiments, the method 800 includes receiving (804) a physiological signal. In some embodiments, the physiological signal is received from a physiological sensor (e.g., HR, PPG, EMG, temperature sensing, etc.; FIG. 1A) coupled to the circuit board (PCB 318; FIG. 3A). In some embodiments, the physiological signal is a measurement such as a heart rate signal of a user wearing the wrist-wearable device 104 and the physiological sensor is any biosensor such as a heart rate sensor, PPG sensors, etc.

In some embodiments, the method 800 includes determining (806) if a motion-artifact criteria is satisfied. In some embodiments, the motion-artifact criteria is satisfied based on the contact pressure signal. For example, the motion-artifact criteria include one of a number of motion-artifacts included on the contact pressure signal, a lack of a contact pressure signal and thus an IMU signal I needed to be used, etc. The weights determine what portion and how much of a motion-artifact or how many motion-artifacts are filtered from the physiological signal.

In some embodiments, the method 800 includes determining (808) a first motion-artifact adjustments to the physiological signal. In some embodiments, the first motion-artifact adjustments are based on the contact pressure signal. For example, as discussed in FIGS. 5 and 6, the contact pressure signal is representative of the noise/distortion of the physiological signal and thus the contact pressure signal contains motion-artifacts that are representative of the motion-artifacts that need to be removed from the physiological signal. The motion-artifacts within the contact pressure signal are used as weights for the adaptive algorithm 508 and the adaptive filter 506 described further in FIG. 5.

In some embodiments, the method 800 includes generating (810) a first motion-artifact compensated physiological signal. In some embodiments, the first motion-artifact compensated physiological signal is generated based on the first motion-artifact adjustments. For example, as described in FIG. 5, after the physiological signal has gone through the adaptive filter 508 and adaptive algorithm 510 a motion-artifact compensated physiological signal is generated.

In some embodiments, the method 800 includes determining (812) if a second motion-artifact criteria is satisfied. In some embodiments, additional motion-artifact criteria are used such as whether or not the contact pressure signal is strong enough and/or contains enough motion-artifacts to filter from the physiological signal.

In some embodiments, the method 800 includes determining (814) second motion-artifact adjustments to the physiological signal. For example, as discussed in FIGS. 5 and 6, the adaptive algorithm 506 is weighted with a plurality of motion-artifacts (e.g., including the first and second motion-artifacts). Adjustments to the physiological signal are made based on the adaptive filter 506 and adaptive algorithm 508 and the determined weights.

In some embodiments, the method 800 includes generating (816) a second motion-artifact compensated physiological signal. The motion-artifact compensated physiological signal (e.g., the contact pressure compensated physiological signal illustrated in FIG. 5) is a filtered signal that excludes overlapping the motion-artifacts that appear in both the physiological signal and the contact pressure signal.

In some embodiments, the method 800 includes determining (818) physiological measurement. In some embodiments, the physiological measurement is determined based on the first motion-artifact compensated physiological signal or the second motion-artifact compensated physiological signal. For example, as described in FIGS. 5 and 6, the physiological measurement is representative of a biological measurement such as a heart rate, skin temperature, etc. The physiological measurement is generated based on the filtered physiological signal and filtered contact pressure signals described in FIGS. 5 and 6.

(A2) In some embodiments of A1, determining, based on the contact pressure signal, the first motion-artifact adjustments includes determining first weights of an adaptive filter used to filter the physiological signal, and determining, based on the contact pressure signal, the second motion-artifact adjustments includes determining second weights of the adaptive filter used to filter the physiological signal. As described in FIGS. 5 and 6, the contact pressure signal is used to determine the weights for the adaptive filter 506 as the motion-artifacts represented in the contact pressure signal are the same motion-artifacts that are desired to be filtered out of the physiological signal.

(A3) In some embodiments of A1 or A2, the pressure sensor is a strain sensor (e.g., a strain gauge sensor) and the strain sensor is coupled to or disposed on one or more of: a band (e.g., band portion 104b or band portion 104c; FIG. 3A) of the wrist-wearable device (e.g., wrist-wearable device 104; FIG. 1A), the circuit board (e.g., PCB 318; FIG. 3A), a back cover (e.g., back cover 304; FIG. 3A) of the capsule (e.g., capsule 104a; FIG. 1A), and a side portion of the capsule. In some embodiments, the pressure sensor is a strain gauge coupled to a portion of the capsule 104a or a PCB 318 within the capsule 104a. The strain gauge measures the displacement of the force to generate the pressure sensing signal.

(A4) In some embodiments of any of A1-A3, the strain sensor is coupled to the circuit board and the contact pressure signal is generated by the strain sensor measuring a strain applied to the circuit board. For example, if the tightness of the band of the wrist-wearable device gets tighter, the additional force exerted to the back cover of the capsule will be passed through to the circuit board coupled to the back cover and ultimately sensed by the strain sensor coupled to the circuit board.

(A5) In some embodiments of any of A1-A4, the pressure sensor is one of a plurality of pressure sensors coupled to the circuit board and each pressure sensor of the plurality of pressure sensors provides a respective contact pressure signals. Additionally, respective motion-artifact adjustments to the physiological signal are determined based on the respective contact pressure signals. As discussed in FIGS. 5 and 6, multiple pressure sensors are coupled to the PCB 318 and the signals generated from the pressure sensors are the contact pressure signals used to determine the weights for the adaptive filter 506 and ultimately the motion-artifacts that need to be removed from the physiological signal.

(A6) In some embodiments of any of A1-A5, the method further includes receiving inertial measurement unit (IMU) data from an IMU of the wrist-wearable device and respective motion-artifact adjustments to the physiological signal are further determined based on the IMU data. As discussed in FIGS. 5 and 6, the filtered physiological signal generated can be input into an algorithm 512 which can include IMU data to further filter the signal from additional motion-artifacts if required.

(A7) In some embodiments of any of A1-A6, the contact pressure signal is representative of a band tightness of the wrist-wearable device when donned by the user. In some embodiments, the band tightness affects the contact pressure signal. For example, the tighter the band on the wrist-wearable device is, the more contact pressure is exhibited on the back cover and thus there is a stronger contract pressure signal. The inverse is also true; the looser the band the lower the contract pressure is exhibited on the back cover of the capsule.

(A8) In some embodiments of any of A1-A7, further including in accordance with a determination that the band tightness of the wrist-wearable device satisfies a predetermined tightness threshold, causing an indication to be presented at the wrist-wearable device, the indication recommending an adjustment to a band of the wrist-wearable device. For example, if the wrist-wearable device 104 is too tight, it can affect the sensor output as described further in FIG. 7.

(A9) In some embodiments of any of A1-A8, in accordance with a determination that the physiological measurement is outside of a predetermined threshold, generating an indication displayed at the wrist-wearable device to adjust a wrist-wearable device band tightness. For example, as discussed in FIG. 7, if the band of the wrist-wearable device 104 is too loose, the measurements may be affected and the capsule can display a message to a user to adjust the band tightness (e.g., make it tighter, or looser).

(B1) In accordance with some embodiments, a non-transitory computer readable storage medium including executable instructions that, when executed by one or more processors, cause the one or more processors to perform or cause performance of one or more operations. The one or more operations include: (i) receiving a contact pressure signal from a pressure sensor coupled to a circuit board within a capsule of a wrist-wearable device donned by a user and (ii) receiving a physiological signal from a physiological sensor coupled to the circuit board. In accordance with a determination, based on the contact pressure signal, that first motion artifact criteria is satisfied, the one or more operations include: (i) determining, based on the contact pressure signal, first motion-artifact adjustments to the physiological signal, and generating, based on the first motion-artifact adjustments, a first motion-artifact compensated physiological signal. In accordance with a determination, based on the contact pressure signal, that second motion artifact criteria is satisfied, the one or more operations include: (i) determining, based on the contact pressure signal, second motion-artifact adjustments to the physiological signal, and (ii) generating, based on the first motion-artifact adjustments, a second motion-artifact compensated physiological signal. The one or more operations further include determining a physiological measurement based on the first motion-artifact compensated physiological signal or the second motion-artifact compensated physiological signal.

(B2) In some embodiments of B1, determining, based on the contact pressure signal, the first motion-artifact adjustments includes determining first weights of an adaptive filter used to filter the physiological signal, and determining, based on the contact pressure signal, the second motion-artifact adjustments includes determining second weights of the adaptive filter used to filter the physiological signal. As described in FIGS. 5 and 6, the contact pressure signal is used to determine the weights for the adaptive filter 506 as the motion-artifacts represented in the contact pressure signal are the same motion-artifacts that are desired to be filtered out of the physiological signal.

(B3) In some embodiments of B1 or B2, the pressure sensor is a strain sensor (e.g., a strain gauge sensor) and the strain sensor is coupled to or disposed on one or more of: a band (e.g., band portion 104b or band portion 104c; FIG. 3A) of the wrist-wearable device (e.g., wrist-wearable device 104; FIG. 1A), the circuit board (e.g., PCB 318; FIG. 3A), a back cover (e.g., back cover 304; FIG. 3A) of the capsule (e.g., capsule 104a; FIG. 1A), and a side portion of the capsule. In some embodiments, the pressure sensor is a strain gauge coupled to a portion of the capsule 104a or a PCB 318 within the capsule 104a. The strain gauge measures the displacement of the force to generate the pressure sensing signal.

(B4) In some embodiments of any of B1-B3, the strain sensor is coupled to the circuit board and the contact pressure signal is generated by the strain sensor measuring a strain applied to the circuit board. For example, if the tightness of the band of the wrist-wearable device gets tighter, the additional force exerted to the back cover of the capsule will be passed through to the circuit board coupled to the back cover and ultimately sensed by the strain sensor coupled to the circuit board.

(B5) In some embodiments of any of B1-B4, the pressure sensor is one of a plurality of pressure sensors coupled to the circuit board and each pressure sensor of the plurality of pressure sensors provides a respective contact pressure signals. Additionally, respective motion-artifact adjustments to the physiological signal are determined based on the respective contact pressure signals. As discussed in FIGS. 5 and 6, multiple pressure sensors are coupled to the PCB 318 and the signals generated from the pressure sensors are the contact pressure signals used to determine the weights for the adaptive filter 506 and ultimately the motion-artifacts that need to be removed from the physiological signal.

(B6) In some embodiments of any of B1-B5, the method further includes receiving inertial measurement unit (IMU) data from an IMU of the wrist-wearable device and respective motion-artifact adjustments to the physiological signal are further determined based on the IMU data. As discussed in FIGS. 5 and 6, the filtered physiological signal generated can be input into an algorithm 512 which can include IMU data to further filter the signal from additional motion-artifacts if required.

(B7) In some embodiments of any of B1-B6, the contact pressure signal is representative of a band tightness of the wrist-wearable device when donned by the user. In some embodiments, the band tightness affects the contact pressure signal. For example, the tighter the band on the wrist-wearable device is, the more contact pressure is exhibited on the back cover and thus there is a stronger contract pressure signal. The inverse is also true; the looser the band the lower the contract pressure is exhibited on the back cover of the capsule.

(B8) In some embodiments of any of B1-B7, further including in accordance with a determination that the band tightness of the wrist-wearable device satisfies a predetermined tightness threshold, causing an indication to be presented at the wrist-wearable device, the indication recommending an adjustment to a band of the wrist-wearable device. For example, if the wrist-wearable device 104 is too tight, it can affect the sensor output as described further in FIG. 7.

(B9) In some embodiments of any of B1-B8, in accordance with a determination that the physiological measurement is outside of a predetermined threshold, generating an indication displayed at the wrist-wearable device to adjust a wrist-wearable device band tightness. For example, as discussed in FIG. 7, if the band of the wrist-wearable device 104 is too loose, the measurements may be affected and the capsule can display a message to a user to adjust the band tightness (e.g., make it tighter, or looser).

(C1) A wrist-wearable device including a capsule including a backplate portion configured to couple to a wrist of a user; a circuit board within the capsule of the wrist-wearable device; and one or more processors including one or more programs, the one or more programs comprising instructions, which, when executed by the wrist-wearable device, cause the wrist-wearable device to perform one or more operations. The one or more operations include: (i) receiving a contact pressure signal from a pressure sensor coupled to a circuit board within a capsule of a wrist-wearable device donned by a user and (ii) receiving a physiological signal from a physiological sensor coupled to the circuit board. In accordance with a determination, based on the contact pressure signal, that first motion artifact criteria is satisfied, the one or more operations include: (i) determining, based on the contact pressure signal, first motion-artifact adjustments to the physiological signal, and generating, based on the first motion-artifact adjustments, a first motion-artifact compensated physiological signal. In accordance with a determination, based on the contact pressure signal, that second motion artifact criteria is satisfied, the one or more operations include: (i) determining, based on the contact pressure signal, second motion-artifact adjustments to the physiological signal, and (ii) generating, based on the first motion-artifact adjustments, a second motion-artifact compensated physiological signal. The one or more operations further include determining a physiological measurement based on the first motion-artifact compensated physiological signal or the second motion-artifact compensated physiological signal.

(C2) In some embodiments of C1, determining, based on the contact pressure signal, the first motion-artifact adjustments includes determining first weights of an adaptive filter used to filter the physiological signal, and determining, based on the contact pressure signal, the second motion-artifact adjustments includes determining second weights of the adaptive filter used to filter the physiological signal. As described in FIGS. 5 and 6, the contact pressure signal is used to determine the weights for the adaptive filter 506 as the motion-artifacts represented in the contact pressure signal are the same motion-artifacts that are desired to be filtered out of the physiological signal.

(C3) In some embodiments of C1 or C2, the pressure sensor is a strain sensor (e.g., a strain gauge sensor) and the strain sensor is coupled to or disposed on one or more of: a band (e.g., band portion 104b or band portion 104c; FIG. 3A) of the wrist-wearable device (e.g., wrist-wearable device 104; FIG. 1A), the circuit board (e.g., PCB 318; FIG. 3A), a back cover (e.g., back cover 304; FIG. 3A) of the capsule (e.g., capsule 104a; FIG. 1A), and a side portion of the capsule. In some embodiments, the pressure sensor is a strain gauge coupled to a portion of the capsule 104a or a PCB 318 within the capsule 104a. The strain gauge measures the displacement of the force to generate the pressure sensing signal.

(C4) In some embodiments of any of C1-C3, the strain sensor is coupled to the circuit board and the contact pressure signal is generated by the strain sensor measuring a strain applied to the circuit board. For example, if the tightness of the band of the wrist-wearable device gets tighter, the additional force exerted to the back cover of the capsule will be passed through to the circuit board coupled to the back cover and ultimately sensed by the strain sensor coupled to the circuit board.

(C5) In some embodiments of any of C1-C4, the pressure sensor is one of a plurality of pressure sensors coupled to the circuit board and each pressure sensor of the plurality of pressure sensors provides a respective contact pressure signals. Additionally, respective motion-artifact adjustments to the physiological signal are determined based on the respective contact pressure signals. As discussed in FIGS. 5 and 6, multiple pressure sensors are coupled to the PCB 318 and the signals generated from the pressure sensors are the contact pressure signals used to determine the weights for the adaptive filter 506 and ultimately the motion-artifacts that need to be removed from the physiological signal.

(C6) In some embodiments of any of C1-C5, the method further includes receiving inertial measurement unit (IMU) data from an IMU of the wrist-wearable device and respective motion-artifact adjustments to the physiological signal are further determined based on the IMU data. As discussed in FIGS. 5 and 6, the filtered physiological signal generated can be input into an algorithm 512 which can include IMU data to further filter the signal from additional motion-artifacts if required.

(C7) In some embodiments of any of C1-C6, the contact pressure signal is representative of a band tightness of the wrist-wearable device when donned by the user. In some embodiments, the band tightness affects the contact pressure signal. For example, the tighter the band on the wrist-wearable device is, the more contact pressure is exhibited on the back cover and thus there is a stronger contract pressure signal. The inverse is also true; the looser the band the lower the contract pressure is exhibited on the back cover of the capsule.

(C8) In some embodiments of any of C1-C7, further including in accordance with a determination that the band tightness of the wrist-wearable device satisfies a predetermined tightness threshold, causing an indication to be presented at the wrist-wearable device, the indication recommending an adjustment to a band of the wrist-wearable device. For example, if the wrist-wearable device 104 is too tight, it can affect the sensor output as described further in FIG. 7.

(C9) In some embodiments of any of C1-C8, in accordance with a determination that the physiological measurement is outside of a predetermined threshold, generating an indication displayed at the wrist-wearable device to adjust a wrist-wearable device band tightness. For example, as discussed in FIG. 7, if the band of the wrist-wearable device 104 is too loose, the measurements may be affected and the capsule can display a message to a user to adjust the band tightness (e.g., make it tighter, or looser).

(D1) In accordance with some embodiments, a system that includes a wrist-wearable device (or a plurality of wrist-wearable devices) and a pair of augmented-reality glasses, and the system is configured to perform operations corresponding to any of A1-C9.

The devices described above are further detailed below, including wrist-wearable devices, headset devices, systems, and haptic feedback devices. Specific operations described above may occur as a result of specific hardware, such hardware is described in further detail below. The devices described below are not limiting and features on these devices can be removed or additional features can be added to these devices.

### Example Extended-Reality Systems

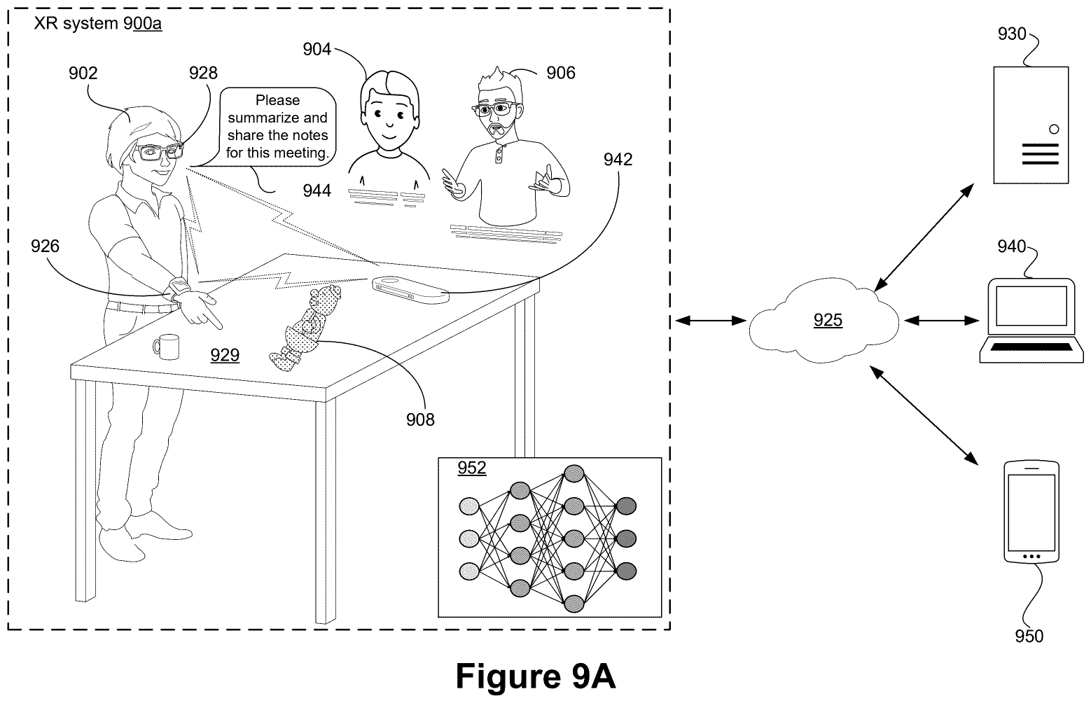

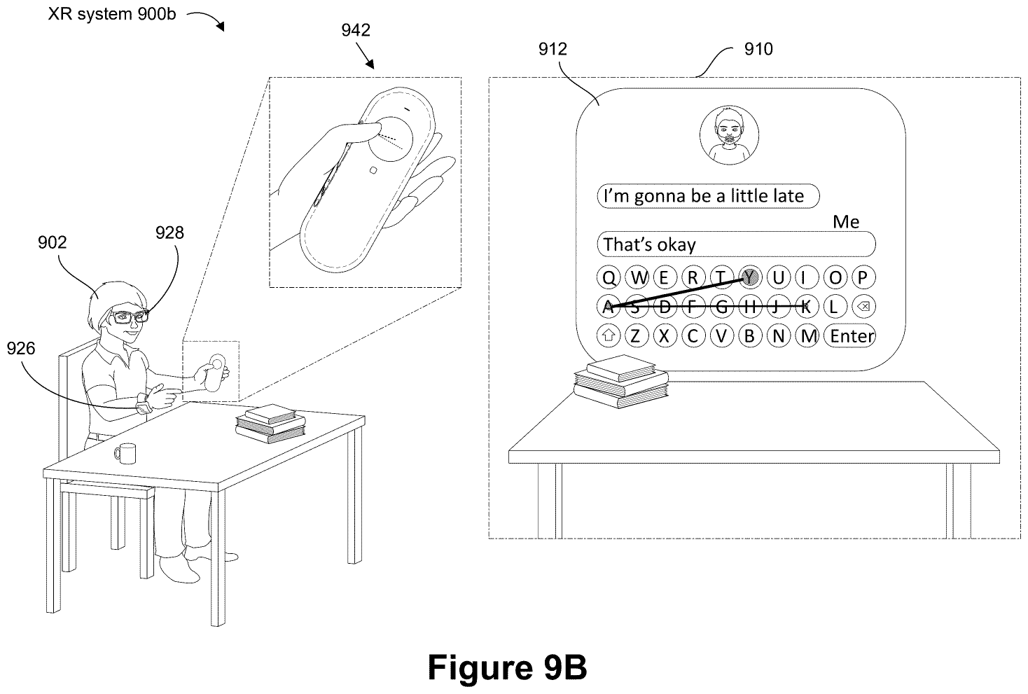

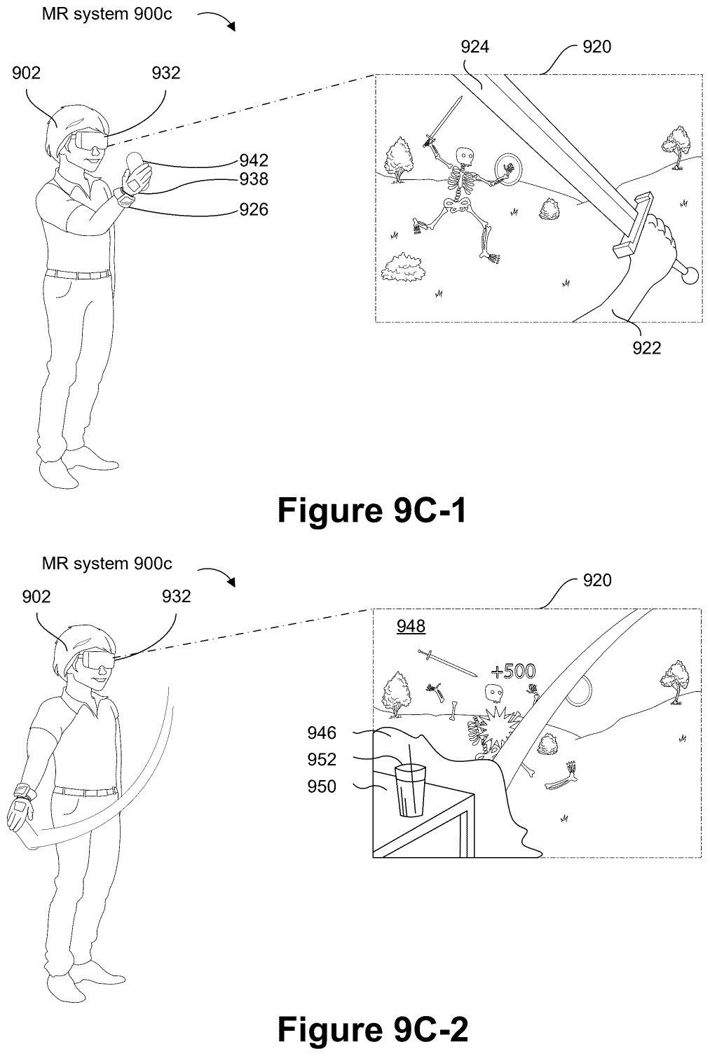

FIGS. 9A, 9B, 9C-1, and 9C-2, illustrate example XR systems that include AR and MR systems, in accordance with some embodiments. FIG. 9A shows a first XR system 900a and first example user interactions using a wrist-wearable device 926, a head-wearable device (e.g., AR device 928), and/or a HIPD 942. FIG. 9B shows a second XR system 900b and second example user interactions using a wrist-wearable device 926, AR device 928, and/or an HIPD 942. FIGS. 9C-1 and 9C-2 show a third MR system 900c and third example user interactions using a wrist-wearable device 926, a head-wearable device (e.g., an MR device such as a VR device), and/or an HIPD 942. As the skilled artisan will appreciate upon reading the descriptions provided herein, the above-example AR and MR systems (described in detail below) can perform various functions and/or operations.

The wrist-wearable device 926, the head-wearable devices, and/or the HIPD 942 can communicatively couple via a network 925 (e.g., cellular, near field, Wi-Fi, personal arca network, wireless LAN). Additionally, the wrist-wearable device 926, the head-wearable device, and/or the HIPD 942 can also communicatively couple with one or more servers 930, computers 940 (e.g., laptops, computers), mobile devices 950 (e.g., smartphones, tablets), and/or other electronic devices via the network 925 (e.g., cellular, near field, Wi-Fi, personal area network, wireless LAN). Similarly, a smart textile-based garment, when used, can also communicatively couple with the wrist-wearable device 926, the head-wearable device(s), the HIPD 942, the one or more servers 930, the computers 940, the mobile devices 950, and/or other electronic devices via the network 925 to provide inputs.

Turning to FIG. 9A, a user 902 is shown wearing the wrist-wearable device 926 and the AR device 928 and having the HIPD 942 on their desk. The wrist-wearable device 926, the AR device 928, and the HIPD 942 facilitate user interaction with an AR environment. In particular, as shown by the first AR system 900a, the wrist-wearable device 926, the AR device 928, and/or the HIPD 942 cause presentation of one or more avatars 904, digital representations of contacts 906, and virtual objects 908. As discussed below, the user 902 can interact with the one or more avatars 904, digital representations of the contacts 906, and virtual objects 908 via the wrist-wearable device 926, the AR device 928, and/or the HIPD 942. In addition, the user 902 is also able to directly view physical objects in the environment, such as a physical table 929, through transparent lens(es) and waveguide(s) of the AR device 928. Alternatively, an MR device could be used in place of the AR device 928 and a similar user experience can take place, but the user would not be directly viewing physical objects in the environment, such as table 929, and would instead be presented with a virtual reconstruction of the table 929 produced from one or more sensors of the MR device (e.g., an outward facing camera capable of recording the surrounding environment).

The user 902 can use any of the wrist-wearable device 926, the AR device 928 (e.g., through physical inputs at the AR device and/or built-in motion tracking of a user's extremities), a smart-textile garment, externally mounted extremity tracking device, the HIPD 942 to provide user inputs, etc. For example, the user 902 can perform one or more hand gestures that are detected by the wrist-wearable device 926 (e.g., using one or more EMG sensors and/or IMUs built into the wrist-wearable device) and/or AR device 928 (e.g., using one or more image sensors or cameras) to provide a user input. Alternatively, or additionally, the user 902 can provide a user input via one or more touch surfaces of the wrist-wearable device 926, the AR device 928, and/or the HIPD 942, and/or voice commands captured by a microphone of the wrist-wearable device 926, the AR device 928, and/or the HIPD 942. The wrist-wearable device 926, the AR device 928, and/or the HIPD 942 include an artificially intelligent digital assistant to help the user in providing a user input (e.g., completing a sequence of operations, suggesting different operations or commands, providing reminders, confirming a command). For example, the digital assistant can be invoked through an input occurring at the AR device 928 (e.g., via an input at a temple arm of the AR device 928). In some embodiments, the user 902 can provide a user input via one or more facial gestures and/or facial expressions. For example, cameras of the wrist-wearable device 926, the AR device 928, and/or the HIPD 942 can track the user 902′s eyes for navigating a user interface.

The wrist-wearable device 926, the AR device 928, and/or the HIPD 942 can operate alone or in conjunction to allow the user 902 to interact with the AR environment. In some embodiments, the HIPD 942 is configured to operate as a central hub or control center for the wrist-wearable device 926, the AR device 928, and/or another communicatively coupled device. For example, the user 902 can provide an input to interact with the AR environment at any of the wrist-wearable device 926, the AR device 928, and/or the HIPD 942, and the HIPD 942 can identify one or more back-end and front-end tasks to cause the performance of the requested interaction and distribute instructions to cause the performance of the one or more back-end and front-end tasks at the wrist-wearable device 926, the AR device 928, and/or the HIPD 942. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 942 can perform the back-end tasks and provide the wrist-wearable device 926 and/or the AR device 928 operational data corresponding to the performed back-end tasks such that the wrist-wearable device 926 and/or the AR device 928 can perform the front-end tasks. In this way, the HIPD 942, which has more computational resources and greater thermal headroom than the wrist-wearable device 926 and/or the AR device 928, performs computationally intensive tasks and reduces the computer resource utilization and/or power usage of the wrist-wearable device 926 and/or the AR device 928.

In the example shown by the first AR system 900a, the HIPD 942 identifies one or more back-end tasks and front-end tasks associated with a user request to initiate an AR video call with one or more other users (represented by the avatar 904 and the digital representation of the contact 906) and distributes instructions to cause the performance of the one or more back-end tasks and front-end tasks. In particular, the HIPD 942 performs back-end tasks for processing and/or rendering image data (and other data) associated with the AR video call and provides operational data associated with the performed back-end tasks to the AR device 928 such that the AR device 928 performs front-end tasks for presenting the AR video call (e.g., presenting the avatar 904 and the digital representation of the contact 906).

In some embodiments, the HIPD 942 can operate as a focal or anchor point for causing the presentation of information. This allows the user 902 to be generally aware of where information is presented. For example, as shown in the first AR system 900a, the avatar 904 and the digital representation of the contact 906 are presented above the HIPD 942. In particular, the HIPD 942 and the AR device 928 operate in conjunction to determine a location for presenting the avatar 904 and the digital representation of the contact 906. In some embodiments, information can be presented within a predetermined distance from the HIPD 942 (e.g., within five meters). For example, as shown in the first AR system 900a, virtual object 908 is presented on the desk some distance from the HIPD 942. Similar to the above example, the HIPD 942 and the AR device 928 can operate in conjunction to determine a location for presenting the virtual object 908. Alternatively, in some embodiments, presentation of information is not bound by the HIPD 942. More specifically, the avatar 904, the digital representation of the contact 906, and the virtual object 908 do not have to be presented within a predetermined distance of the HIPD 942. While an AR device 928 is described working with an HIPD, an MR headset can be interacted with in the same way as the AR device 928.

User inputs provided at the wrist-wearable device 926, the AR device 928, and/or the HIPD 942 are coordinated such that the user can use any device to initiate, continue, and/or complete an operation. For example, the user 902 can provide a user input to the AR device 928 to cause the AR device 928 to present the virtual object 908 and, while the virtual object 908 is presented by the AR device 928, the user 902 can provide one or more hand gestures via the wrist-wearable device 926 to interact and/or manipulate the virtual object 908. While an AR device 928 is described working with a wrist-wearable device 926, an MR headset can be interacted with in the same way as the AR device 928.

### Integration of Artificial Intelligence With XR Systems

FIG. 9A illustrates an interaction in which an artificially intelligent virtual assistant can assist in requests made by a user 902. The AI virtual assistant can be used to complete open-ended requests made through natural language inputs by a user 902. For example, in FIG. 9A the user 902 makes an audible request 944 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 9A also illustrates an example neural network 952 used in Artificial Intelligence applications. Uses of Artificial Intelligence (AI) are varied and encompass many different aspects of the devices and systems described herein. AI capabilities cover a diverse range of applications and deepen interactions between the user 902 and user devices (e.g., the AR device 928, an MR device 932, the HIPD 942, the wrist-wearable device 926). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user's request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 902 can interact with an AI model through natural language inputs captured by a voice sensor, text inputs, or any other input modality that accepts natural language and/or a corresponding voice sensor module. In another instance, input is provided by tracking the eye gaze of a user 902 via a gaze tracker module. Additionally, the AI model can also receive inputs beyond those supplied by a user 902. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors' data can be retrieved entirely from a single device (e.g., AR device 928) or from multiple devices that are in communication with each other (e.g., a system that includes at least two of an AR device 928, an MR device 932, the HIPD 942, the wrist-wearable device 926, etc.). The AI model can also access additional information (e.g., one or more servers 930, the computers 940, the mobile devices 950, and/or other electronic devices) via a network 925.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 928, an MR device 932, the HIPD 942, the wrist-wearable device 926) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 928, an MR device 932, the HIPD 942, the wrist-wearable device 926), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 942), haptic feedback can provide information to the user 902. An Al model can also use the inputs described above to determine the appropriate modality and device(s) to present content to the user (e.g., a user walking on a busy road can be presented with an audio output instead of a visual output to avoid distracting the user 902).

### Example Augmented Reality Interaction

FIG. 9B shows the user 902 wearing the wrist-wearable device 926 and the AR device 928 and holding the HIPD 942. In the second AR system 900b, the wrist-wearable device 926, the AR device 928, and/or the HIPD 942 are used to receive and/or provide one or more messages to a contact of the user 902. In particular, the wrist-wearable device 926, the AR device 928, and/or the HIPD 942 detect and coordinate one or more user inputs to initiate a messaging application and prepare a response to a received message via the messaging application.

In some embodiments, the user 902 initiates, via a user input, an application on the wrist-wearable device 926, the AR device 928, and/or the HIPD 942 that causes the application to initiate on at least one device. For example, in the second AR system 900b the user 902 performs a hand gesture associated with a command for initiating a messaging application (represented by messaging user interface 912); the wrist-wearable device 926 detects the hand gesture; and, based on a determination that the user 902 is wearing the AR device 928, causes the AR device 928 to present a messaging user interface 912 of the messaging application. The AR device 928 can present the messaging user interface 912 to the user 902 via its display (e.g., as shown by user 902's field of view 910). In some embodiments, the application is initiated and can be run on the device (e.g., the wrist-wearable device 926, the AR device 928, and/or the HIPD 942) that detects the user input to initiate the application, and the device provides another device operational data to cause the presentation of the messaging application. For example, the wrist-wearable device 926 can detect the user input to initiate a messaging application, initiate and run the messaging application, and provide operational data to the AR device 928 and/or the HIPD 942 to cause presentation of the messaging application. Alternatively, the application can be initiated and run at a device other than the device that detected the user input. For example, the wrist-wearable device 926 can detect the hand gesture associated with initiating the messaging application and cause the HIPD 942 to run the messaging application and coordinate the presentation of the messaging application.

Further, the user 902 can provide a user input provided at the wrist-wearable device 926, the AR device 928, and/or the HIPD 942 to continue and/or complete an operation initiated at another device. For example, after initiating the messaging application via the wrist-wearable device 926 and while the AR device 928 presents the messaging user interface 912, the user 902 can provide an input at the HIPD 942 to prepare a response (e.g., shown by the swipe gesture performed on the HIPD 942). The user 902's gestures performed on the HIPD 942 can be provided and/or displayed on another device. For example, the user 902's swipe gestures performed on the HIPD 942 are displayed on a virtual keyboard of the messaging user interface 912 displayed by the AR device 928.

In some embodiments, the wrist-wearable device 926, the AR device 928, the HIPD 942, and/or other communicatively coupled devices can present one or more notifications to the user 902. The notification can be an indication of a new message, an incoming call, an application update, a status update, etc. The user 902 can select the notification via the wrist-wearable device 926, the AR device 928, or the HIPD 942 and cause presentation of an application or operation associated with the notification on at least one device. For example, the user 902 can receive a notification that a message was received at the wrist-wearable device 926, the AR device 928, the HIPD 942, and/or other communicatively coupled device and provide a user input at the wrist-wearable device 926, the AR device 928, and/or the HIPD 942 to review the notification, and the device detecting the user input can cause an application associated with the notification to be initiated and/or presented at the wrist-wearable device 926, the AR device 928, and/or the HIPD 942.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 928 can present to the user 902 game application data and the HIPD 942 can use a controller to provide inputs to the game. Similarly, the user 902 can use the wrist-wearable device 926 to initiate a camera of the AR device 928, and the user can use the wrist-wearable device 926, the AR device 928, and/or the HIPD 942 to manipulate the image capture (e.g., zoom in or out, apply filters) and capture image data.

While an AR device 928 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user's hand or other suitable surface (e.g., a table, whiteboard). In yet another embodiment, information may also be provided by locally dimming portions of a lens to emphasize portions of the environment in which the user's attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 9C-1 and 9C-2, the user 902 is shown wearing the wrist-wearable device 926 and an MR device 932 (e.g., a device capable of providing either an entirely VR experience or an MR experience that displays object(s) from a physical environment at a display of the device) and holding the HIPD 942. In the third AR system 900c, the wrist-wearable device 926, the MR device 932, and/or the HIPD 942 are used to interact within an MR environment, such as a VR game or other MR/VR application. While the MR device 932 presents a representation of a VR game (e.g., first MR game environment 920) to the user 902, the wrist-wearable device 926, the MR device 932, and/or the HIPD 942 detect and coordinate one or more user inputs to allow the user 902 to interact with the VR game.

In some embodiments, the user 902 can provide a user input via the wrist-wearable device 926, the MR device 932, and/or the HIPD 942 that causes an action in a corresponding MR environment. For example, the user 902 in the third MR system 900c (shown in FIG. 9C-1) raises the HIPD 942 to prepare for a swing in the first MR game environment 920. The MR device 932, responsive to the user 902 raising the HIPD 942, causes the MR representation of the user 922 to perform a similar action (e.g., raise a virtual object, such as a virtual sword 924). In some embodiments, each device uses respective sensor data and/or image data to detect the user input and provide an accurate representation of the user 902's motion. For example, image sensors (e.g., SLAM cameras or other cameras) of the HIPD 942 can be used to detect a position of the HIPD 942 relative to the user 902's body such that the virtual object can be positioned appropriately within the first MR game environment 920; sensor data from the wrist-wearable device 926 can be used to detect a velocity at which the user 902 raises the HIPD 942 such that the MR representation of the user 922 and the virtual sword 924 are synchronized with the user 902's movements; and image sensors of the MR device 932 can be used to represent the user 902's body, boundary conditions, or real-world objects within the first MR game environment 920.

In FIG. 9C-2, the user 902 performs a downward swing while holding the HIPD 942. The user 902's downward swing is detected by the wrist-wearable device 926, the MR device 932, and/or the HIPD 942 and a corresponding action is performed in the first MR game environment 920. In some embodiments, the data captured by each device is used to improve the user's experience within the MR environment. For example, sensor data of the wrist-wearable device 926 can be used to determine a speed and/or force at which the downward swing is performed and image sensors of the HIPD 942 and/or the MR device 932 can be used to determine a location of the swing and how it should be represented in the first MR game environment 920, which, in turn, can be used as inputs for the MR environment (e.g., game mechanics, which can use detected speed, force, locations, and/or aspects of the user 902′s actions to classify a user's inputs (e.g., user performs a light strike, hard strike, critical strike, glancing strike, miss) or calculate an output (e.g., amount of damage)).

FIG. 9C-2 further illustrates that a portion of the physical environment is reconstructed and displayed at a display of the MR device 932 while the MR game environment 920 is being displayed. In this instance, a reconstruction of the physical environment 946 is displayed in place of a portion of the MR game environment 920 when object(s) in the physical environment are potentially in the path of the user (e.g., a collision with the user and an object in the physical environment are likely). Thus, this example MR game environment 920 includes (i) an immersive VR portion 948 (e.g., an environment that does not have a corollary counterpart in a nearby physical environment) and (ii) a reconstruction of the physical environment 946 (e.g., table 929 and cup). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 926, the MR device 932, and/or the HIPD 942 are described as detecting user inputs, in some embodiments, user inputs are detected at a single device (with the single device being responsible for distributing signals to the other devices for performing the user input). For example, the HIPD 942 can operate an application for generating the first MR game environment 920 and provide the MR device 932 with corresponding data for causing the presentation of the first MR game environment 920, as well as detect the user 902's movements (while holding the HIPD 942) to cause the performance of corresponding actions within the first MR game environment 920. Additionally or alternatively, in some embodiments, operational data (e.g., sensor data, image data, application data, device data, and/or other data) of one or more devices is provided to a single device (e.g., the HIPD 942) to process the operational data and cause respective devices to perform an action associated with processed operational data.

In some embodiments, the user 902 can wear a wrist-wearable device 926, wear an MR device 932, wear smart textile-based garments 938 (e.g., wearable haptic gloves), and/or hold an HIPD 942 device. In this embodiment, the wrist-wearable device 926, the MR device 932, and/or the smart textile-based garments 938 are used to interact within an MR environment (e.g., any AR or MR system described above in reference to FIGS. 9A-9B). While the MR device 932 presents a representation of an MR game (e.g., second MR game environment 920) to the user 902, the wrist-wearable device 926, the MR device 932, and/or the smart textile-based garments 938 detect and coordinate one or more user inputs to allow the user 902 to interact with the MR environment.

In some embodiments, the user 902 can provide a user input via the wrist-wearable device 926, an HIPD 942, the MR device 932, and/or the smart textile-based garments 938 that causes an action in a corresponding MR environment. In some embodiments, each device uses respective sensor data and/or image data to detect the user input and provide an accurate representation of the user 902′s motion. While four different input devices are shown (e.g., a wrist-wearable device 926, an MR device 932, an HIPD 942, and a smart textile-based garment 938) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 938) sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user's experience within the MR environment. Although not shown, the smart textile-based garments 938 can be used in conjunction with an MR device and/or an HIPD 942.

While some experiences are described as occurring on an AR device and other experiences are described as occurring on an MR device, one skilled in the art would appreciate that experiences can be ported over from an MR device to an AR device, and vice versa.

### Other Interactions

While numerous examples are described in this application related to extended-reality environments, one skilled in the art would appreciate that certain interactions may be possible with other devices. For example, a user may interact with a robot (e.g., a humanoid robot, a task specific robot, or other type of robot) to perform tasks inclusive of, leading to, and/or otherwise related to the tasks described herein. In some embodiments, these tasks can be user specific and learned by the robot based on training data supplied by the user and/or from the user's wearable devices (including head-worn and wrist-wearable, among others) in accordance with techniques described herein. As one example, this training data can be received from the numerous devices described in this application (e.g., from sensor data and user-specific interactions with head-wearable devices, wrist-wearable devices, intermediary processing devices, or any combination thereof). Other data sources are also conceived outside of the devices described here. For example, AI models for use in a robot can be trained using a blend of user-specific data and non-user specific-aggregate data. The robots may also be able to perform tasks wholly unrelated to extended reality environments, and can be used for performing quality-of-life tasks (e.g., performing chores, completing repetitive operations, etc.). In certain embodiments or circumstances, the techniques and/or devices described herein can be integrated with and/or otherwise performed by the robot.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

The foregoing descriptions of FIGS. 9A-9C-2 provided above are intended to augment the description provided in reference to FIGS. 1A-8. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if”' can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A method comprising:
receiving a contact pressure signal from a pressure sensor coupled to a circuit board within a capsule of a wrist-wearable device donned by a user;
receiving a physiological signal from a physiological sensor coupled to the circuit board; and
in accordance with a determination, based on the contact pressure signal, that a first motion-artifact criteria is satisfied:
determining, based on the contact pressure signal, first motion-artifact adjustments to the physiological signal, and
p2 generating, based on the first motion-artifact adjustments, a first motion-artifact compensated physiological signal;
in accordance with a determination, based on the contact pressure signal, that second motion artifact criteria is satisfied:
determining, based on the contact pressure signal, second motion-artifact adjustments to the physiological signal, and
generating, based on the first motion-artifact adjustments, a second motion-artifact compensated physiological signal; and
determining a physiological measurement based on the first motion-artifact compensated physiological signal or the second motion-artifact compensated physiological signal.

2. The method of claim 1, wherein:
determining, based on the contact pressure signal, the first motion-artifact adjustments includes determining first weights of an adaptive filter used to filter the physiological signal; and
determining, based on the contact pressure signal, the second motion-artifact adjustments includes determining second weights of the adaptive filter used to filter the physiological signal.

3. The method of claim 1, wherein:
the pressure sensor is a strain sensor; and
the strain sensor is coupled to or disposed on one or more of: a band of the wrist-wearable device, the circuit board, a back cover of the capsule, and a side portion of the capsule.

4. The method of claim 3, wherein:
the strain sensor is coupled to the circuit board and the contact pressure signal is generated by the strain sensor measuring a strain applied to the circuit board.

5. The method of claim 1, wherein:
the pressure sensor is one of a plurality of pressure sensors coupled to the circuit board;
each pressure sensor of the plurality of pressure sensors provides a respective contact pressure signals; and
respective motion-artifact adjustments to the physiological signal are determined based on the respective contact pressure signals.

6. The method of claim 1, further comprising:
receiving inertial measurement unit (IMU) data from an IMU of the wrist-wearable device; and
wherein respective motion-artifact adjustments to the physiological signal are further determined based on the IMU data.

7. The method of claim 1, wherein the contact pressure signal is representative of a band tightness of the wrist-wearable device when donned by the user.

8. The method of claim 7, further comprising:
in accordance with a determination that the band tightness of the wrist-wearable device satisfies a predetermined tightness threshold, causing an indication to be presented at the wrist-wearable device, the indication recommending an adjustment to a band of the wrist-wearable device.

9. The method of claim 1, further including:
in accordance with a determination that the physiological measurement is outside of a predetermined threshold, generating an indication displayed at the wrist-wearable device to adjust a wrist-wearable device band tightness.

10. A non-transitory, computer-readable storage medium including executable instructions that, when executed by one or more processors, cause the one or more processors to perform or cause performance of:
receiving a contact pressure signal from a pressure sensor coupled to a circuit board within a capsule of a wrist-wearable device donned by a user;
receiving a physiological signal from a physiological sensor coupled to the circuit board; and
in accordance with a determination, based on the contact pressure signal, that first motion artifact criteria is satisfied:
determining, based on the contact pressure signal, first motion-artifact adjustments to the physiological signal, and
generating, based on the first motion-artifact adjustments, a first motion-artifact compensated physiological signal;
in accordance with a determination, based on the contact pressure signal, that second motion artifact criteria is satisfied:
determining, based on the contact pressure signal, second motion-artifact adjustments to the physiological signal, and
generating, based on the first motion-artifact adjustments, a second motion-artifact compensated physiological signal; and
determining a physiological measurement based on the first motion-artifact compensated physiological signal or the second motion-artifact compensated physiological signal.

11. The non-transitory, computer-readable storage medium of claim 10, wherein:
determining, based on the contact pressure signal, the first motion-artifact adjustments includes determining first weights of an adaptive filter used to filter the physiological signal; and
determining, based on the contact pressure signal, the second motion-artifact adjustments includes determining second weights of the adaptive filter used to filter the physiological signal.

12. The non-transitory, computer-readable storage medium of claim 10, wherein:
the pressure sensor is a strain sensor; and
the strain sensor is coupled to or disposed on one or more of: a band of the wrist-wearable device, the circuit board, a back cover of the capsule, and a side portion of the capsule.

13. The non-transitory, computer-readable storage medium of claim 12, wherein:
the strain sensor is coupled to the circuit board and the contact pressure signal is generated by the strain sensor measuring a strain applied to the circuit board.

14. The non-transitory, computer-readable storage medium of claim 10, wherein:
the pressure sensor is one of a plurality of pressure sensors coupled to the circuit board;
each pressure sensor of the plurality of pressure sensors provides a respective contact pressure signals; and
respective motion-artifact adjustments to the physiological signal are determined based on the respective contact pressure signals.

15. A wrist-wearable device comprising:
a capsule including a backplate portion configured to couple to a wrist of a user;
a circuit board within the capsule of the wrist-wearable device; and
one or more processors including one or more programs, the one or more programs comprising instructions, which, when executed by the wrist-wearable device, cause the wrist-wearable device to:
receive a contact pressure signal from a pressure sensor coupled to the circuit board;
receive a physiological signal from a physiological sensor coupled to the circuit board; and
in accordance with a determination, based on the contact pressure signal, that first motion artifact criteria is satisfied:
determine, based on the contact pressure signal, first motion-artifact adjustments to the physiological signal, and
generate, based on the first motion-artifact adjustments, a first motion-artifact compensated physiological signal;
in accordance with a determination, based on the contact pressure signal, that second motion artifact criteria is satisfied:
determine, based on the contact pressure signal, second motion-artifact adjustments to the physiological signal, and
generate, based on the first motion-artifact adjustments, a second motion-artifact compensated physiological signal; and
determine a physiological measurement based on the first motion-artifact compensated physiological signal or the second motion-artifact compensated physiological signal.

16. The wrist-wearable device of claim 15, wherein:
determining, based on the contact pressure signal, the first motion-artifact adjustments includes determining first weights of an adaptive filter used to filter the physiological signal; and
determining, based on the contact pressure signal, the second motion-artifact adjustments includes determining second weights of the adaptive filter used to filter the physiological signal.

17. The wrist-wearable device of claim 15, wherein:
the pressure sensor is a strain sensor; and
the strain sensor is coupled to or disposed on one or more of: a band of the wrist-wearable device, the circuit board, a back cover of the capsule, and a side portion of the capsule.

18. The wrist-wearable device of claim 17, wherein:
the strain sensor is coupled to the circuit board and the contact pressure signal is generated by the strain sensor measuring a strain applied to the circuit board.

19. The wrist-wearable device of claim 15, wherein:
the pressure sensor is one of a plurality of pressure sensors coupled to the circuit board;
each pressure sensor of the plurality of pressure sensors provides a respective contact pressure signals; and
respective motion-artifact adjustments to the physiological signal are determined based on the respective contact pressure signals.

20. The wrist-wearable device of claim 15, further including:
in accordance with a determination that the physiological measurement is outside of a predetermined threshold, generating an indication displayed at the wrist-wearable device to adjust a wrist-wearable device band tightness.
